(19) United States
(12) Patent Application Publication
(10) Pub. No.: US 2021/0108276 A1
(43) Pub. Date: Apr. 15, 2021

(54) TUMOR-ASSOCIATED MARKERS FOR DETECTION OF MINIMAL RESIDUAL DISEASE USING DIGITAL DROPLET PCR

(71) Applicant: CHILDREN'S NATIONAL MEDICAL CENTER, Washington, DC (US)

(72) Inventors: Kirsten WILLIAMS, Atlanta, GA (US); Melanie GRANT, Silver Spring, MD (US); Javad NAZARIAN, Zurich (CH); Maja STANOJEVIC, Silver Spring, MD (US)

(73) Assignee: CHILDREN'S NATIONAL MEDICAL CENTER, Washington, DC (US)

(21) Appl. No.: 17/132,176

(22) Filed: Dec. 23, 2020

Related U.S. Application Data
(63) Continuation of application No. PCT/US2019/039281, filed on Jun. 26, 2019.
(60) Provisional application No. 62/690,170, filed on Jun. 26, 2018.

(51) Int. Cl. C12Q 1/6886

(52) U.S. Cl. CPC C12Q 1/6886; C12Q 2600/158; C12Q 2600/118

(57) ABSTRACT

The invention relates to a digital droplet polymerase chain reaction method that simultaneously detects and can quantify relative levels of multiple tumor associated antigens associated with relapse of a cancer after prior treatment. It does not require invasive bone marrow sampling or biopsy and permits rational targeting based on the types of TAAs expressed by relapsing cancer cells.

### CROSS-REFERENCE TO RELATED APPLICATION

This application is a continuation of PCT/US2019/039281, filed Jun. 26, 2019, which claims priority to U.S. Provisional Application No. 62/690,170, filed Jun. 26, 2018. Both of these applications are hereby incorporated by reference for all purposes.

### REFERENCE TO A SEQUENCE LISTING

In accordance with 37 CFR § 1.52(ex5), the present specification makes reference to a Sequence Listing (submitted electronically as a .txt file named “534537US_ST25.txt”. The .txt file was generated on Dec. 22, 2020 and is 5.89 kb in size. The entire contents of the Sequence Listing are herein incorporated by reference.

### BACKGROUND OF THE INVENTION

### Field of the Invention

The invention falls within the fields of medical diagnostics and therapeutics, especially within the fields of immunology and oncology.

### Description of the Related Art

Relapse of a cancer, tumor, or neoplasm after cancer treatment is a significant indicator of treatment failure and of a need for supplemental treatment, more robust treatment, or different cancer treatment regimen, such as a retargeted immunotherapy directed at different antigens expressed by a cancer. Cancer relapse results from persistence of neoplastic cells that have not been destroyed by prior treatments including cancer or tumor cells resistant to radiation, chemotherapy, or immunotherapy.

Unfortunately for previously treated cancer patients, relapse is often insidious as relapsing cancer cells can be present in a patient in undetectable numbers especially in the early stages of a relapse when further treatment would be the most beneficial. For example, undetectable residual numbers of malignant cells may remain in a patient's blood after treatment for leukemia or lymphoma, such as after ablation and hematopoietic stem cell transplant.

Relapse is the leading cause of death in such patients as shown in FIG. 1 and the risk of relapse of a primary cancer exceeds 30%. Prognosis after relapse is dismal with fewer than 10% of relapsing patients surviving.

Currently, there is insufficient information about the phenotypes of relapsing cells. Much is not known about whether relapsing cells express particular kinds, levels, or ratios of tumor-associated antigens such as BIRC5, PRAME, or WT1. Despite the identification of tumor-associated antigens (“TAAs”) associated with blood cancers, the use of these TAAs for diagnosis or as targets for further treatment of minimal residual disease or early relapse of a tumor has not been evaluated.

Tumor antigens associated with leukemia and lymphoma include BIRC5 (Survivin), PRAME (preferentially expressed antigen in melanoma) and WT1 (Wilms tumor 1). Representative polynucleotide sequences of WT1, Survivin, and PRAME are incorporated by reference to the following reference sequences. A reference gene encoding Survivin (BIRC5) is described by NCBI Reference Sequence: NG_029069.1. A reference gene encoding PRAME is described by HGNC 9336. A reference gene encoding WT1 is described by HGNC 12796. Amino acid sequences for BIRC5, PRAME, and WT1 are also described by these reference sequences or may be easily deduced from them using the genetic code. The terms WT1, Survivin, and PRAME as used herein include all known human homologs of these genes or proteins as of Jun. 26, 2019.

Tumor associated antigens WT1, BIRC5 (Survivin), and PRAME are over-expressed in up to 80-100% of AML (acute myeloid leukemia), MDS (myelodysplastic syndromes), CML (chromic myeloid leukemia), ALL (acute lymphoblastic leukemia), HL (Hodgkin's lymphoma) and ALCL (anaplastic large-cell lymphoma) cells.

The presence or expression of a tumor associated antigen may be identified by detection of its RNA or DNA or by detection of the TAA per se, for example, by a TAA-specific antibody. Biological samples useful for detecting TAAs include blood, plasma, cell-free plasma or serum, or of a cellular fraction of blood or bone marrow. All or part of a TAA gene, coding sequence of a TAA or TAA mRNA may be detected, for example, by dd-PCR.

Circulating immunity to TAAs often correlates with disease responses and early identification of TAAs associated with minimal residual disease or relapse may help guide targeted immunotherapy; Melenhorst, et al., High avidity myeloid leukemia-associated antigen-specific CD8⁺ T cells preferentially reside in the bone marrow, Blood 2009 113:2238-2244; doi: https://_doi.org/10.1182/blood-2008-04-151969; Rezvani, et al., Characterizing and optimizing immune responses to leukaemia antigens after allogeneic stem cell transplantation, Blood, September 2008, Volume 21, Issue 3, Pages 437-453; and Troeger, et al., Survivin and its prognostic significance in pediatric acute B-cell precursor lymphoblastic leukemia, Haematologica, 2007 August; 92(8):1043-50. Epub 2007 Jul. 20; and can be measured using PCR of WT1, see FIG. 2.

To be effective a targeted immunotherapy should specifically target a TAA expressed by cancer cells. However, existing methods for detecting TAA in leukemia and lymphoma suffer from a number of problems. The current standard of care involves taking bone marrow aspirates or biopsies which is painful, costly and invasive. Moreover, these procedures often miss recovering diseased cells because leukemia may occur in patches and not detectable in a bone marrow sample. Many types of leukemia lack specific cancer markers and existing methods for detection of leukemia cells by have limited specificity and sensitivity when disease burden is low (as in minimal residual disease) and when markers are also present on healthy cells. Consequently, there is a need for more specific and sensitive tests for early detection of leukemia and other blood cancer cells especially in patients having minimal residual disease after prior anticancer therapy and at risk for relapse. A highly sensitive and specific test that could identify a subject at risk for cancer relapse prior to or in the very early stages of relapse and help pick out a targeted immunotherapy for cancer cells would be invaluable.

The polymerase chain reaction (“PCR”) can be used to detect nucleic acids from TAAs. Digital droplet PCR is a very sensitive method for detection of RNA copy numbers, permitting enumeration of tumor antigen RNA copy numbers. Digital drop PCR is described by Jennings, el al., J. Mol. Diagnos. 16(2), March 2014; real-time quantitative RT-PCR to study preferentially expressed antigen of melanoma (“PRAME”) by Rezvani, et al., Blood 113(10), Mar. 5, 2009; and real time quantitative PCR by Cilloni, et al., Leukemia 16:2115-2121 (2002).

So far this procedure has only been applied to detecting a single tumor associated antigen, BCR-ABL, but not other blood cancer antigens and it has not been used to simultaneously detect and quantify more than one tumor associated antigen in a single procedure.

While PCR provides a way to detect tumor associated antigens in blood, existing methods suffer from several limitations. Conventional RT-PCR does not simultaneously detect levels of RNA for multiple TAA markers and from a control marker such as ABL. This makes it difficult or impossible to compare RNA translation levels of each TAA marker with a control marker. RT-PCR is also limited by its detection threshold and by its requirement for a threshold number of cancer cells.

In view of the problems with detecting multiple TAA markers in the blood without the need for biopsy or bone marrow collection, the inventors sought to develop a simple, noninvasive method that uses a blood or plasma sample to simultaneously determine and compare levels of multiple TAA markers. Such a method can simultaneously detect multiple tumor antigen copy numbers, as well as copy numbers of control gene(s) with the objective of providing the quantitative data that sensitively identifies minimal residual disease burden, risk of relapse, and to help guide subsequent targeted immunotherapy, such as tumor-associated antigen-specific lymphocyte (“TAA-L”) based therapies targeting BIRC5, PRAME or WT1.

### SUMMARY OF THE INVENTION

The invention is generally directed to a digital droplet polymerase chain reaction (“dd-PCR”) method that simultaneously detects levels of multiple TAAs. This method may be performed on RNA or DNA samples obtained non-invasively from blood or blood products and does not require a bone marrow sample or biopsy.

The invention also identifies specific TAA markers for minimal residual disease (“MRD”) associated with cancer relapse thus providing for early detection and targeted retreatment of relapsing cancer.

In other embodiments, the invention is directed to compositions of cancer cell markers useful for detecting and characterizing by digital droplet PCT. These compositions may comprise two or more cancer cell markers selected for assessment of a particular cancer, for example, for chronic myelogenous leukemia markers for BCR-ABL, WT1, PRAME, and BIRC-5 status may be used to perform ddPCR and to derive an expression profile for cancer in a particular subject. An expression profile may take into account whether a mutation identified by a particular marker is present or take into account its level of expression in a subject.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Causes of death of leukemia patients after treatment (pie chart).

FIG. 2. Example of use of PCR to follow T cell numbers and track tumor markers.

FIG. 3. A model of targeted immunotherapy involving expansion of T cells that recognize tumor antigens and subsequent infusion of these T cells in to cancer patient.

FIG. 4. Normalized BCR-ABL tumor cell marker levels as determined by RT-PCR. Patient was treated with Dasatinib and subsequently with TAA-L resulting in remission of cancer to levels below a threshold of detection. Dasatinib is a chemotherapy that targets kinases such as Bcr-Abl and Src kinase family kinases and is used to treat CML and ALL and cancers that are Philadelphia chromosome positive.

FIG. 5. Amplification plot showing that RNA encoding BIRC5, PRAME, WT1 and BCR-ABL can be individually amplified by PCR.

FIG. 6. Example of use of dd-PCR to detect tumor antigen RNA in plasma and direct further immunological treatment based on quantification of marker.

FIGS. 7A-7C. Detection of tumor associated antigens BIRC5, PRAME and WT1 in cell lines expressing these antigens and in a buffy coat sample spiked with tumor cells. FIG. 7A: No template control; FIG. 7B: TAA cell lines; FIG. 7C: TAA cell lined spiked buffy coat.

FIGS. 7D-7F. Detection of tumor associated antigens BIRC5, PRAME and WT1 in cell lines expressing these antigens and in a buffy coat sample spiked with tumor cells. FIG. 7D: No template control; FIG. 7E: TAA cell line+buffy coat; FIG. 7F: BIRC5+ABL1.

FIG. 8A. Negative dd-PCR control.

FIG. 8B. Positive control showing dd-PCR detection of WT1, PRAME and BIRC5 RNA.

FIG. 9. RNA levels for WT1, BIRC5 and PRAME normalized to control ABL1 levels.

FIG. 10A. Negative dd-PCR control.

FIG. 10B. Positive dd-PCR control.

FIG. 10C. Detection of WT1 and BIRC5, but not PRAME.

FIG. 10D. Detection of WT1 and BIRC5, but not PRAME at 3 weeks post-treatment.

FIG. 10E. Decrease in level of BIRC5 RNA compared at baseline and 3 weeks post-treatment.

FIG. 11A shows lower baseline values for WT1 and BIRC5 (Survivin) compared to values 3 weeks post-treatment shown in FIG. 11B.

FIG. 11B. Tumor marker values at 3 weeks post treatment.

FIG. 11C. BIRC5 and WT1 values increased as measured 3 weeks post-treatment.

FIG. 12A. Week 2 values for levels of tumor associated antigen RNA.

FIG. 12B. Week 5 values for levels of tumor associated antigen RNA.

FIG. 12C. Levels of BIRC5 (Survivin) decreased 5 weeks post-treatment, but levels of WT1 increased suggesting that treatment did not adequately target WT1.

FIG. 13A. Patient tumor associated antigen RNA levels pre-treatment.

FIG. 13B. Patient tumor associated antigen RNA levels before death and TAA-L treatment. High levels of all BIRC5, WT1 and PRAME were detected.

FIG. 14. Table showing relative levels of tumor antigen RNAs normalized to ABL control.

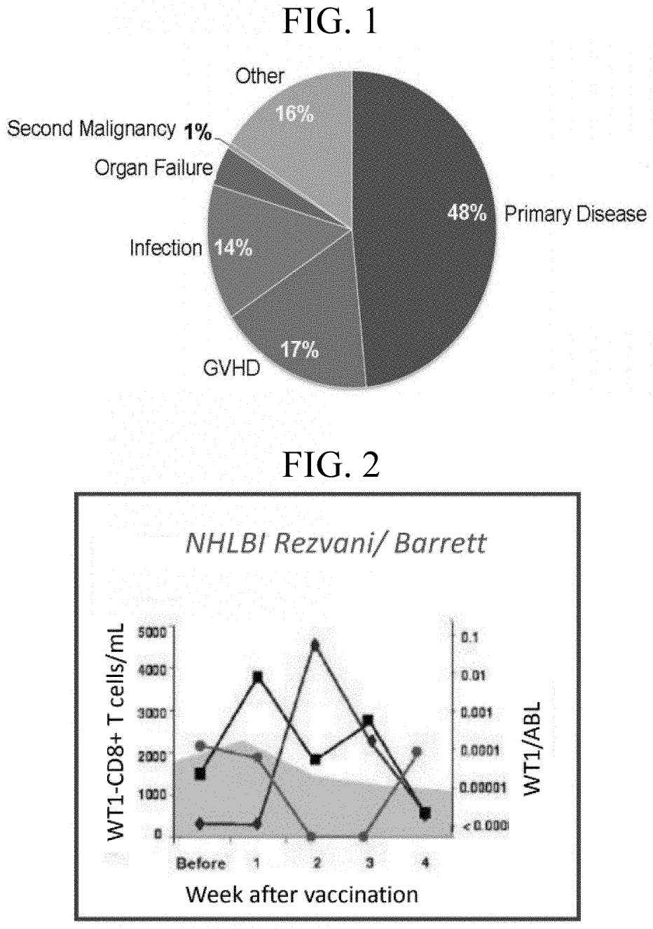

### DETAILED DESCRIPTION OF THE INVENTION

The diagnostic and therapeutic methods of the invention may be used after, in conjunction with, or before a cancer treatment. Typically, it is used after a cancer treatment for an earlier identification of cancer relapse and the specific cancer markers associated with the relapse.

Many different cancer treatments are known including surgery, chemotherapy, radiation therapy, biological or immunotherapy. Therapies also include treatment with hormones, cytokines or other biological response modifiers, or targeted therapy using monoclonal antibodies to tumor associated antigens or that use T cells that recognize tumor associated antigens. Those skilled in the art may select a kind of treatment or combination of treatments depending on the type of cancer and patient being treated. Those treatments may be assessed and monitored using the methods described herein.

Treatments may be modified depending on the results obtained using the methods of the invention, for example, a level of a chemotherapy or immunotherapy targeted at cancer cells expressing particular TAAs may be increased, decreased or discontinued based on the information obtained regarding tumor associated antigens in a relapsing cancer. In some instances a therapy may be retargeted at one or more TAAs associated with the relapse that may or may not have been targeted by a prior treatment. The effects of these and other modes of cancer therapy including on status of minimal residual disease or status of relapse, may be assessed and treated using methods according to the invention.

Immunotherapies for cancer include but are not limited to:

Adoptive cell transfer is a treatment that attempts to boost the natural ability of a subject's T cells to fight cancer. T cells are a type of white blood cell and part of the immune system. T cells active against cancer may be isolated from a tumor or location containing tumor cells or may be recovered from a subject or a donor and sensitized in vitro. T cells reactive against tumor antigens may be expanded in the laboratory and then reinfused or infused into a cancer patient, for example, by intravenous infusion. Examples of T cells that may be used in the invention include those that are induce to, recognize and/or target TAAs such as BIRC5, PRAME and/or WT1 or other known cancer antigens or cancer antigens disclosed herein. Preferably, T cells will target antigens that characterize a cancer relapse.

TAA-L therapy involves infusion or adoptive transfer of tumor-associated antigen-specific lymphocytes (“TAA-L”) which may be autologous (e.g., derived from lymphocytes, stem cells or cord blood cells of a subject being treated) or allogenic from a donor with a different genetic background such as a parent, child, sibling or other close relative. Examples of T cells that may be used in TAA-L therapy according to the invention include those that are induced to, recognize and/or target TAAs such as BIRC5, PRAME and/or WT1 or other known cancer antigens or cancer antigens disclosed herein. Preferably, T cells will target antigens that characterize a cancer relapse.

CAR T cell therapy involves infusion or transfer of CAR T cells which are T cells engineered to express special receptors on their surfaces and then expanded in vitro prior to return to a subject being treated. CAR T cell therapy is further described and incorporated by reference to the National Cancer Institute at https://_www.cancer.gov/about-cancer/treatment/research/car-t-cells (last accessed Apr. 27, 2018). Examples of CAR T cells that may be used in therapy according to the invention include those that are engineered to express receptors that recognize and/or target TAAs such as BIRC5, PRAME and/or WT1 or other known cancer antigens or cancer antigens disclosed herein.

Monoclonal antibody therapy. Monoclonal antibodies drugs that are designed to bind to specific targets in the body such as particular tumor associated antigens. Once bound to a tumor they may induce or promote an immune response that destroys the cancer cells Monoclonal antibodies can also be used to “mark” cancer cells so it is easier for the immune system to find and destroy them.

Cytokine therapy. Cytokines are proteins that are made by a subject's cells and play important roles in the body's normal immune responses and also in the immune system's ability to respond to cancer. The two main types of cytokines used to treat cancer are called interferons and interleukins.

Vaccination. This involves vaccinating the subject with tumor-associated immunogens that boost a subject's immune system response to cancer cells. An anti-cancer vaccine can be cell-based, protein- or peptide-based, or gene-based (e.g., based on natural or modified DNA/RNA).

BCG administration. BCG stands for Bacillus Calmette-Guérin, is an immunotherapeutic that is used induce an immune response against cancer cells.

Other immunotherapies are described by, and incorporated by reference to, https://en.wikipedia.org/wiki/Cancer_immunotherapy#Adoptive_T-cell_therapy.

Chemotherapies for cancers include those using the drugs described by, and incorporated by reference to https://_www.cancer.gov/about-cancer/treatment/drugs/cancer-type (last accessed Apr. 28, 2018).

Leukemia treatment prior to, in conjunction with, or after performing a diagnostic method according to the invention may include one or more of the following.

Chemotherapy. Chemotherapy is a major form of treatment for leukemia. This type of drug treatment uses chemicals to kill leukemia cells. Depending on the type of leukemia a subject has, a single drug or a combination of drugs may be received. These drugs may come in a pill form, or they may be injected directly into a vein.

Biological therapy. Biological therapy works by using treatments that help a subject's immune system recognize and attack leukemia cells, such as the administration of tumor-antigen-specific T cells or antibodies, such as agents that recognize BIRC5, PRAME and/or WT1 TAAs.

Targeted therapy. Targeted therapy uses drugs or immunological agents that attack specific vulnerabilities within a subject's cancer cells. For example, the drug imatinib (Gleevec) stops the action of a protein within the leukemia cells of people with chronic myelogenous leukemia. This can help control the disease.

Radiation therapy. Radiation therapy uses X-rays or other high-energy beams to damage leukemia cells and stop their growth. During radiation therapy, a patient usually lies on a table while a large machine moves around the patient, directing the radiation to precise points on the body. Radiation may be received in one specific area of the body where there is a collection of leukemia cells or whole body radiation may be received. Radiation therapy may be used to prepare for a stem cell transplant Stem cell transplant. A stem cell transplant is a procedure to replace a subject's diseased bone marrow with healthy bone marrow. Before a stem cell transplant, the subject receives high doses of chemotherapy or radiation therapy to destroy diseased bone marrow. Then an infusion of blood-forming stem cells that help to rebuild healthy bone marrow is received. Stem cells may be received from a donor, or in some cases be a subject's own stem cells. A stem cell transplant is very similar to a bone marrow transplant.

These and other therapeutic methods, may be used prior to, in conjunction with, a diagnostic or therapeutic method of the invention. The invention enhances targeted anti-tumor therapies, such as therapies using tumor-associated antigen-specific lymphocytes (“TAA-L”) by revealing which tumor antigens in relapsing tumors are good therapeutic targets.

Drugs approved for treatment of acute lymphoblastic leukemia (“ALL”) include, but are not limited to, Abitrexate (Methotrexate), Arranon (Nelarabine), Asparaginase Erwinia chrysanthemi, Besponsa (Inotuzumab Ozogamicin), Blinatumomab, Blincyto (Blinatumomab), Cerubidine (Daunorubicin Hydrochloride), Clafen (Cyclophosphamide), Clofarabine, Clofarex (Clofarabine), Clolar (Clofarabine), Cyclophosphamide, Cytarabine, Cytosar-U (Cytarabine), Cytoxan (Cyclophosphamide), Dasatinib, Daunorubicin Hydrochloride, Doxorubicin Hydrochloride, Erwinaze (Asparaginase Erwinia Chrysanthemi), Folex (Methotrexate), Folex PFS (Methotrexate), Gleevec (Imatinib Mesylate), Iclusig (Ponatinib Hydrochloride), Inotuzumab Ozogamicin, Imatinib Mesylate, Kymriah (Tisagenlecleucel), Marqibo (Vincristine Sulfate Liposome), Mercaptopurine, Methotrexate, Methotrexate LPF (Methorexate), Mexate (Methotrexate), Mexate-AQ (Methotrexate), Nelarabine, Neosar (Cyclophosphamide), Oncaspar (Pegaspargase), Pegaspargase, Ponatinib Hydrochloride, Prednisone, Purinethol (Mercaptopurine), Purixan (Mercaptopurine), Rubidomycin (Daunorubicin Hydrochloride), Sprycel (Dasatinib), Tarabine PFS (Cytarabine), Tisagenlecleucel, Vincasar PFS (Vincristine Sulfate), Vincristine Sulfate, and Vincristine Sulfate Liposome.

Combination therapies for acute lymphoblastic leukemia (“ALL”) which may be used prior to or in conjunction with a diagnostic or therapeutic method of the invention include Hyper-CVAD. Such drugs and combination therapies are further described by the National Cancer Institute at https://_www.cancer.gov/about-cancer/treatment/drugs/leukemia (incorporated by reference, incorporated by reference, last accessed Apr. 27, 2018).

Drugs approved for treatment of acute myeloid leukemia (“AML”) include, but are not limited to, Arsenic Trioxide, Cerubidine (Daunorubicin Hydrochloride), Clafen (Cyclophosphamide), Cyclophosphamide, Cytarabine, Cytosar-U (Cytarabine), Cytoxan (Cyclophosphamide), Daunorubicin Hydrochloride, Daunorubicin Hydrochloride and Cytarabine Liposome, Doxorubicin Hydrochloride, Enasidenib Mesylate, Idamycin (Idarubicin Hydrochloride), Idarubicin Hydrochloride, Idhifa (Enasidenib Mesylate), Midostaurin, Mitoxantrone Hydrochloride, Neosar (Cyclophosphamide), Rubidomycin (Daunorubicin Hydrochloride), Rydapt (Midostaurin), Tabloid (Thioguanine), Tarabine PFS (Cytarabine), Thioguanine, Trisenox (Arsenic Trioxide), Vincasar PFS (Vincristine Sulfate), Vincristine Sulfate, and Vyxeos (Daunorubicin Hydrochloride and Cytarabine Liposome).

Combination therapies for AML which may be used prior to or in conjunction with a diagnostic or therapeutic method of the invention include ADE. Such drugs and combination therapies are further described by the National Cancer Institute at https://_www.cancer.gov/about-cancer/treatment/drugs/leukemia (incorporated by reference, incorporated by reference, last accessed Apr. 27, 2018).

Drugs approved for treatment of Chronic Lymphocytic Leukemia (CLL) include, but are not limited to, Alemtuzumab, Ambochlorin (Chlorambucil), Amboclorin (Chlorambucil), Arzerra (Ofatumumab), Bendamustine Hydrochloride, Campath (Alemtuzumab), Chlorambucil, Clafen (Cyclophosphamide), Cyclophosphamide, Cytoxan (Cyclophosphamide), Fludara (Fludarabine Phosphate), Fludarabine Phosphate, Gazyva (Obinutuzumab), Ibrutinib, Idelalisib, Imbruvica (Ibrutinib), Leukeran (Chlorambucil), Linfolizin (Chlorambucil), Mechlorethamine Hydrochloride, Mustargen (Mechlorethamine Hydrochloride), Neosar (Cyclophosphamide), Obinutuzumab, Ofatumumab, Prednisone Rituxan (Rituximab), Rituxan Hycela (Rituximab and Hyaluronidase Human), Rituximab, Rituximab and Hyaluronidase Human, Treanda (Bendamustine Hydrochloride), Venclexta (Venetoclax), Venetoclax, and Zydelig (Idelalisib).

Combination therapies for CLL which may be used prior to or in conjunction with a diagnostic or therapeutic method of the invention include CHLORAMBUCIL-PREDNISONE and CVP (Cyclophosphamide-Vincristine Sulfate-Prednisone). Such drugs and combination therapies are further described by the National Cancer Institute at https://_www.cancer.gov/about-cancer/treatment/drugs/leukemia (incorporated by reference, incorporated by reference, last accessed Apr. 27, 2018).

Drugs approved for treatment of Chronic Myelogenous Leukemia (CML) include, but are not limited to, Bosulif (Bosutinib), Bosutinib, Busulfan, Busulfex (Busulfan), Clafen (Cyclophosphamide), Cyclophosphamide, Cytarabine, Cytosar-U (Cytarabine), Cytoxan (Cyclophosphamide), Dasatinib, Gleevec (Imatinib Mesylate), Hydrea (Hydroxyurea), Hydroxyurea, Iclusig (Ponatinib Hydrochloride), Imatinib Mesylate, Mechlorethamine Hydrochloride, Mustargen (Mechlorethamine Hydrochloride), Myleran (Busulfan), Neosar (Cyclophosphamide), Nilotinib, Omacetaxine Mepesuccinate, Ponatinib Hydrochloride, Sprycel (Dasatinib), Synribo (Omacetaxine Mepesuccinate), Tarabine PFS (Cytarabine), and Tasigna (Nilotinib).

Drugs approved for treatment of Hairy Cell Leukemia include, but are not limited to, Cladribine, Intron A (Recombinant Interferon Alfa-2b), Leustatin (Cladribine) and Recombinant Interferon Alfa-2b. Such drugs are further described by the National Cancer Institute at https://_www.cancer.gov/about-cancer/treatment/drugs/leukemia (incorporated by reference, incorporated by reference, last accessed Apr. 27, 2018).

Drugs approved for treatment of Mast Cell Leukemia include, but are not limited to, Midostaurin and Rydapt (Midostaurin). Such drugs are further described by the National Cancer Institute at https://_www.cancer.gov/about-cancer/treatment/drugs/leukemia (incorporated by reference).

Drugs approved for treatment of Meningeal Leukemia include, but are not limited to, Cytarbine, Cytosar-U (Cytarabine), and Tarabine PFS (Cytarabine). Such drugs are further described by the National Cancer Institute at https://_www.cancer.gov/about-cancer/treatment/drugs/leukemia (incorporated by reference, incorporated by reference, last accessed Apr. 27, 2018).

Lymphoma treatment prior to, in conjunction with, or after performing a diagnostic method according to the invention may include one or more of the following.

Active surveillance. Some forms of lymphoma are very slow growing and treatment may be postponed pending emergence of signs and symptoms that interfere with a subject's daily activities. Until then, a subject may undergo periodic tests to monitor the condition.

Chemotherapy. Chemotherapy uses drugs to destroy fast-growing cells, such as cancer cells. The drugs are usually administered through a vein, but can also be taken as a pill, depending on the specific drugs received.

Other drug therapy. Other drugs used to treat lymphoma include targeted drugs that focus on specific abnormalities in a subject's cancer cells. Immunotherapy drugs use the immune system to kill cancer cells.

Biological therapy. Biological therapy works by using treatments that help a subject's immune system recognize and attack lymphoma cells, such as the administration of tumor-antigen-specific T cells or antibodies. Biological therapies may be used to target or retarget TAAs associated with cancer relapse such as relapsing cancer cells expressing BIRC5, PRAME, and/or WT1.

Targeted therapy. Targeted therapy uses drugs or immunological agents that attack specific vulnerabilities within a subject's cancer cells.

Radiation therapy. Radiation therapy uses high-powered beams of energy, such as X-rays and protons, to kill cancer cells.

Bone marrow transplant. A bone marrow transplant, also known as a stem cell transplant, involves using high doses of chemotherapy and radiation to suppress a subject's bone marrow. Then autologous or allogenic healthy bone marrow stem cells from the subject's body or from a donor are infused into a subject's blood where they travel to bones and rebuild healthy bone marrow. These and other therapeutic methods may be used prior to, in conjunction with, a diagnostic or therapeutic method of the invention.

Drugs approved for treatment of Hodgkin's lymphoma include, but are not limited to, Adcetris (Brentuximab Vedotin), Ambochlorin (Chlorambucil), Amboclorin (Chlorambucil), Becenum (Carmustine), BiCNU (Carmustine), Blenoxane (Bleomycin), Bleomycin, Brentuximab Vedotin, Carmubris (Carmustine), Carmustine, Chlorambucil, Clafen (Cyclophosphamide), Cyclophosphamide, Cytoxan (Cyclophosphamide), Dacarbazine, Doxorubicin Hydrochloride, DTIC-Dome (Dacarbazine), Keytruda (Pembrolizumab), Leukeran (Chlorambucil), Linfolizin (Chlorambucil), Lomustine, Matulane (Procarbazine Hydrochloride), Mechlorethamine Hydrochloride, Mustargen (Mechlorethamine Hydrochloride), Neosar (Cyclophosphamide), Nivolumab, Opdivo (Nivolumab), Pembrolizumab, Prednisone, Procarbazine Hydrochloride, Velban (Vinblastine Sulfate), Velsar (Vinblastine Sulfate), Vinblastine Sulfate, Vincasar PFS (Vincristine Sulfate), and Vincristine Sulfate. One or more of these drugs may be used prior to or in conjunction with the diagnostic and therapeutic methods of the invention.

Combination therapies for Hodgkin's lymphoma which may be used prior to or in conjunction with the invention include ABVD, ABVE, ABVE-PC, BEACOPP, COPDAC, COPP, COPP-ABV, ICE, MOPP, OEPA, STANDFORD V, and VAMP. Such drugs and combination therapies are further described by the National Cancer Institute at https://_www.cancer.gov/about-cancer/treatment/drugs/hodgkin-lymphoma (incorporated by reference, last accessed Apr. 27, 2018).

Drugs approved for treatment of non-Hodgkin's lymphoma include, but are not limited to, Abitrexate (Methotrexate), Acalabrutinib, Adcetris (Brentuximab Vedotin), Aliqopa (Copanlisib Hydrochloride), Ambochlorin (Chlorambucil), Amboclorin (Chlorambucil), Arranon (Nelarabine), Axicabtagene Ciloleucel, Becenum (Carmustine), Beleodaq (Belinostat), Belinostat, Bendamustine Hydrochloride, BiCNU (Carmustine), Blenoxane (Bleomycin), Bleomycin, Bortezomib, Brentuximab Vedotin, Calquence (Acalabrutinib), Carmubris (Carmustine), Carmustine, Chlorambucil, Clafen (Cyclophosphamide), Copanlisib Hydrochloride, Cyclophosphamide, Cytoxan (Cyclophosphamide), Cytarabine Liposome, Denileukin Diftitox, DepoCyt (Cytarabine Liposome), Dexamethasone, Doxorubicin Hydrochloride, Folex (Methotrexate), Folex PFS (Methotrexate), Folotyn (Pralatrexate), Gazyva (Obinutuzumab), Ibritumomab Tiuxetan, Ibrutinib, Idelalisib, Imbruvica (Ibrutinib), Intron A (Recombinant Interferon Alfa-2b), Istodax (Romidepsin), Lenalidomide, Leukeran (Chlorambucil), Linfolizin (Chlorambucil), Mechlorethamine Hydrochloride, Methotrexate, Methotrexate LPF (Methotrexate), Mexate (Methotrexate), Mexate-AQ (Methotrexate), Mozobil (Plerixafor), Mustargen (Mechlorethamine Hydrochloride), Nelarabine, Neosar (Cyclophosphamide), Obinutuzumab, Ontak (Denileukin Diftitox), Plerixafor, Pralatrexate, Prednisone, Recombinant Interferon Alfa-2b, Revlimid (Lenalidomide), Rituxan (Rituximab), Rituxan Hycela (Rituximab and Hyaluronidase Human), Rituximab, Rituximab and Hyaluronidase Human, Romidepsin, Treanda (Bendamustine Hydrochloride), Velban (Vinblastine Sulfate), Velcade (Bortezomib), Velsar (Vinblastine Sulfate), Vinblastine Sulfate, Vincasar PFS (Vincristine Sulfate), Vincristine Sulfate, Vorinostat, Yescarta (Axicabtagene Ciloleucel), Zevalin (Ibritumomab Tiuxetan), Zolinza (Vorinostat), and Zydelig (Idelalisib). One or more of these drugs may be used prior to or in conjunction with the diagnostic and therapeutic methods of the invention.

Combination therapies for non-Hodgkin lymphoma which may be used prior to or in conjunction with the diagnostic and therapeutic methods of the invention include CHOP, COPP, CVP, EPOCH, Hyper-CVAD, ICE, R-CHOP, R-CVP, R-EPOCH and R-ICE. Such drugs and combination therapies are further described by the National Cancer Institute at https://_www.cancer.gov/about-cancer/treatment/drugs/non-hodgkin (incorporated by reference, incorporated by reference, last accessed Apr. 27, 2018).

The terms diagnosing, detecting, and identifying when used with minimal residual disease (MRD) or undiagnosed cancer are used interchangeably herein to refer to the identifying or detecting cells and/or cell products in specimens that are indicative of disease. Diagnosing also includes staging or identifying a phase of a cancer, such as a leukemia or lymphoma. For example, ALL exhibits several phases including untreated ALL (e.g., more than 5% immature white blood cells or blasts); minimal residual disease (e.g., ALL appears to be in remission but some tests still identify residual leukemia cells in the bone marrow); refractory ALL (e.g., the leukemia persists and is not responding to treatment); and relapsed or recurrent ALL (e.g., the leukemia has returned after a remission).

Non-Hodgkin lymphoma may be staged as Stage I, when the lymphoma is in only 1 lymph node area or lymphoid organ such as the tonsils (I) or when the cancer is found only in 1 area of a single organ outside of the lymph system (IE); as Stage II when the lymphoma is in 2 or more groups of lymph nodes on the same side of (above or below) the diaphragm (the thin band of muscle that separates the chest and abdomen), or when the lymphoma is in a group of lymph node(s) and in one area of a nearby organ (IIE); as Stage III when the lymphoma is in lymph node areas on both sides of (above and below) the diaphragm, or when the lymphoma is in lymph nodes above the diaphragm, as well as in the spleen; and as Stage IV when the lymphoma has spread widely into at least one organ outside the lymph system, such as the bone marrow, liver, or lung.

Hodgkin's Lymphoma may be staged as Stage I when Hodgkin lymphoma is found in only 1 lymph node area or lymphoid organ such as the thymus (I), or when the cancer is found only in 1 area of a single organ outside the lymph system (IE); as Stage II when the lymphoma is found in 2 or more lymph node areas on the same side of (above or below) the diaphragm, which is the thin muscle beneath the lungs that separates the chest and abdomen (II) or when the cancer extends locally from one lymph node area into a nearby organ (IIE); as Stage III when Hodgkin lymphoma is found in lymph node areas on both sides of (above and below) the diaphragm (III), or when lymphoma is in lymph nodes above the diaphragm, as well as in the spleen; and as Stage IV when Hodgkin lymphoma has spread widely into at least one organ outside of the lymph system, such as the liver, bone marrow, or lungs.

The diagnostic and therapeutic methods of the invention may be applied to patients under treatment for various stages or phases of a cancer. Advantageously the methods of the invention are applied to patients who have already been treated, have minimal residual disease, and are at risk of relapse because these methods will guide further treatment of the patient based on the expression of tumor associated antigens or particular levels of or ratios of tumor associated antigens.

Tumor cell markers disclosed herein are markers detectable using PCR. Advantageously the method measures a level of a tumor cell marker using nucleic acids (i.e., RNA or DNA) in a biological sample. Preferably, selected markers will be detectable in blood, buffy coat cells, bone marrow, biopsy tissue, plasma, serum, CSF, tissue fluid, mucus, saliva, urine and biological samples that can be obtained easily or non-invasively, though any biological sample that can be detected and/or quantified using dd-PCR may be used. For example, a method according to the invention can measure the amount of RNA in blood or plasma encoding a TAA marker such as BCR/ABL, WT1, PRAME, and BIRC-5. WT1, PRAME and BIRC5 (Survivin) are highly expressed in many different types of tumors including relapsed or refractory leukemia, lymphomas, neuroblastoma, Wilms tumor, sarcoma and others.

Examples of polynucleotide sequences for ABL1, BIRC5, WT1, and PRAME are described as SEQ ID NOS: 1, 2, 3, and 4, respectively. The invention is also directed to variants of these sequences, such as allelic variants of ABL1, BIRC5, WT1, and PRAME or consensus polynucleotide sequences based on comparison of two or more variant sequences. Probes and primers may be based on these sequences or their complements. Those skilled in the art can select primers that will amplify all or part of a target sequence or probes that bind to all or part of a target sequence.

A reciprocal translocation between chromosomes 22 and 9 produces the Philadelphia chromosome which is often found in patients with chronic myelogenous leukemia. The chromosome 22 breakpoint for this translocation is located within the BCR gene. The translocation produces a fusion protein which is encoded by sequence from both BCR and ABL, the gene at the chromosome 9 breakpoint. Although the BCR-ABL fusion protein has been extensively studied, the function of the normal BCR gene product is not clear. The protein has serine/threonine kinase activity and is a GTPase-activating protein for p21rac. Two transcript variants encoding different isoforms have been found for this gene.

Bcr-Abl tyrosine-kinase inhibitors (TKI) are the first-line therapy for most patients with chronic myelogenous leukemia (CML). More than 90% of CML cases are caused by a chromosomal abnormality that results in the formation of a so-called Philadelphia chromosome. Bcr-Abl tyrosine-kinase inhibitors (TKI) are the first-line therapy for most patients with chronic myelogenous leukemia (CML). More than 90% of CML cases are caused by a chromosomal abnormality that results in the formation of a so-called Philadelphia chromosome. This abnormality was discovered by Peter Nowell in 1960 and is a consequence of fusion between the Abelson (Abl) tyrosine kinase gene at chromosome 9 and the break point cluster (Bcr) gene at chromosome 22, resulting in a chimeric oncogene (Bcr-Abl) and a constitutively active Bcr-Abl tyrosine kinase that has been implicated in the pathogenesis of CML. Compounds have been developed to selectively inhibit the tyrosine kinase.

WT1 or Wilms tumor protein is a protein that in humans is encoded by the WT1 gene on chromosome 11p. This gene encodes a transcription factor that contains four zinc finger motifs at the C-terminus and a proline/glutamine-rich DNA-binding domain at the N-terminus. It has an essential role in the normal development of the urogenital system, and it is mutated in a subset of patients with Wilms' tumor, the gene's namesake. Multiple transcript variants, resulting from alternative splicing at two coding exons, have been well characterized. There is also evidence for the use of non-AUG (CUG) translation initiation site upstream of, and in-frame with the first AUG, leading to additional isoforms. Identifiers: PF01265 (http://pfam.xfam.org/family/PF02165). IPR000976 (https://_www.ebi.ac.uk/interpro/entry/IPR000976). Abnormal expression of the WT1 gene also occurs in some cancers of blood-forming cells (leukemia), such as acute lymphoblastic leukemia (ALL), chronic myeloid leukemia (CML), and childhood acute myeloid leukemia (AML). Links above last accessed Jan. 12, 2018.

PRAME (Melanoma antigen preferentially expressed in tumors) is a protein that in humans is encoded by the PRAME gene. This gene encodes an antigen that is predominantly expressed in human melanomas and that is recognized by cytolytic T lymphocytes. It is not expressed in normal tissues, except testis. This expression pattern is similar to that of other CT antigens, such as MAGE, BAGE and GAGE. However, unlike these other CT antigens, this gene is also expressed in acute leukemia. Five alternatively spliced transcript variants encoding the same protein have been observed for this gene. Interestingly, the PRAME gene is localized between the variable subgenes for light chain of immunoglobulins (Ig), and it is lost in B lymphocytes that rearrange specific lambda variable Ig subgenes. Identifier: PRAME; HGNC:9336 (https://_www.genenames.org/cgi-bin/gene_symbol_report?hgnc_id=9336). Links above last accessed Jan. 12, 2018. PRAME may act as repressor of retinoic acid receptor and likely confers a growth advantage to cancer cells via this function.

Survivin or BIRC5 (baculoviral inhibitor of apoptosis repeat-containing 5) is a member of the inhibitor of apoptosis (IAP) family and is known by identifiers: BIRC5, HGNC:593 (https://_www.genenanes.org/cgi-bin/gene_symbol_report?hgnc_id=593. Human survivin or BIRC5 is a protein that is encoded by the BIRC5 gene, such as that described by NCBI Reference Sequence: NG_029069.1.

Survivin is known to be expressed during fetal development and across most tumor cell types, but is rarely present in normal, non-malignant adult cells. Ambrosini G, Adida C, Altieri DC (1997). “A novel anti-apoptotic gene, survivin, expressed in cancer and lymphoma”. Nat. Med. 3 (8): 917-21. doi:10.1038/nm0897-917. PMID 9256286. Links above last accessed Jan. 12, 2018. This gene is a member of the inhibitor of apoptosis (IAP) gene family, which encode negative regulatory proteins that prevent apoptotic cell death. IAP family members usually contain multiple baculovirus IAP repeat (BIR) domains, but this gene encodes proteins with only a single BIR domain. The encoded proteins also lack a C-terminus RING finger domain. Gene expression is high during fetal development and in most tumors, yet low in adult tissues.

Control genes include ABL (or ABL1), BCR (breakpoint cluster region protein; HGNC 1014) and GUSB (glucuronidase beta; HGNC 4396). These genes may be used as controls, for example, to quantify relative amounts of, or normalize, other TAA nucleic acids detected in a multiplexed sample. ABL or ABL1 (ABL proto-oncogene 1, non-receptor tyrosine kinase; HGNC 76) is a proto-oncogene that encodes a protein tyrosine kinase involved in a variety of cellular processes, including cell division, adhesion, differentiation, and response to stress. It can be used as a control gene to normalize levels of TAA markers detected in a multiplex procedure. The activity of the protein is negatively regulated by its SH3 domain, whereby deletion of the region encoding this domain results in an oncogene. The ubiquitously expressed protein has DNA-binding activity that is regulated by CDCl₂-mediated phosphorylation, suggesting a cell cycle function. This gene has been found fused to a variety of translocation partner genes in various leukemia, most notably the t(9;22) translocation that results in a fusion with the 5′ end of the breakpoint cluster region gene (BCR; MIM:151410). Alternative splicing of this gene results in two transcript variants, which contain alternative first exons that are spliced to the remaining common exons. Other control or housekeeping genes include, but are not limited to Line1, GAPDH, HSP90, β-actin, and β2-microglobulin. In some embodiments, an amount of one or more target TAA polynucleotide sequences may be compared to an amount of one or more control or housekeeping genes.

Other markers. Additional information about tumor markers, biological samples containing tumor markers, and other cancer symptom, diagnosis, prognosis and staging information is incorporated by reference to https://_www.cancer.gov/about-cancer/diagnosis-staging/diagnosis/tumor-markers-fact-sheet (last accessed Jan. 12, 2018).

ALK gene rearrangements and overexpression are associated with non-small cell lung cancer and anaplastic large cell lymphoma

Alpha-fetoprotein (AFP) are associated with liver cancer and germ cell tumors.

Beta-2-microglobulin (B2M) is associated with multiple myeloma, chronic lymphocytic leukemia, and some lymphomas.

Beta-human chorionic gonadotropin (Beta-hCG) is associated with choriocarcinoma and germ cell tumors.

BRCA1 and BRCA2 gene mutations are associated with ovarian cancer.

BCR-ABL fusion gene (Philadelphia chromosome) as also described above is associated with acute lymphoblastic leukemia and acute myelogenous leukemia.

BRAF V600 mutations are associated with cutaneous melanoma and colorectal cancer.

C-kit/CDJ17 is associated with gastrointestinal stromal tumor and mucosal melanoma.

CA15-3/CA27.29 is associated with breast cancer.

CA19-9 is associated with pancreatic cancer, gallbladder cancer, bile duct cancer, and gastric cancer.

CA-125 is associated with ovarian cancer.

Calcitonin is associated with medullary thyroid cancer. RNA encoding calcitonin and other protein markers described herein may be detected using the dd-PCR method of the invention.

Carcinoembryonic antigen (CEA) is associated with colorectal cancer and some other cancers. RNA encoding CEA and other protein markers described herein may be detected using the dd-PCR method of the invention.

CD20 is associated with Hodgkin lymphoma.

Chromogranin A (CgA) is associated with neuroendocrine tumors.

Chromosomes 3, 7, 17, and 9p21 associated with bladder cancer.

Circulating tumor cells of epithelial origin (CELLSEARCH®) associated with metastatic breast, prostate, and colorectal cancers. RNA obtained from circulating tumor may be detected using the dd-PCR method of the invention.

Cytokeratinfragment 21-1 associated with lung cancer.

EGFR gene mutation analysis associated with non-small cell lung cancer.

Estrogen receptor (ER)/progesterone receptor (PR) associated with breast cancer. RNA encoding ER and/or PR described herein may be detected using the dd-PCR method of the invention.

Fibrin/fibrinogen associated with bladder cancer. RNA encoding Fibrin/fibrinogen or abnormal levels thereof may be detected using the dd-PCR method of the invention.

HE4 is associated with ovarian cancer.

HER2/neu gene amplification or protein overexpression are associated with breast cancer, gastric cancer, and gastroesophageal junction adenocarcinoma.

Immunoglobulins are associated with multiple myeloma and Waldenström macroglobulinemia. RNA encoding particular immunoglobulins or abnormal immunoglobulin expression may be detected using the dd-PCR method of the invention.

KRAS gene mutation analysis is associated with colorectal cancer and non-small cell lung cancer.

One or more of these markers may be detected or targeted for treatment in addition to, or instead of, BIRC5, PRAME, or WT1, using a dd-PCR method according to the invention.

Probe/primer combinations may be designed based on known nucleic acid sequences for these TAAs or by methods known in the art such as those described by Busu, PCR Primer Design in Methods in Molecular Biology, 2^{nd} edition, Springer Verlag (2015, incorporated by reference; ISBN 10: 3923645). In some cases specific multiplexing primer/probe sets must be designed to effectively detect two, three, four or more TAA markers by multiplexing. Increases or decreases of these markers, or relative abundance of 2, 3, 4 or more different markers may be correlated with a stage or phase of a cancer, such as relapse after remission. Markers present in relapsing cancers, especially in early stages of relapses, may be specifically targeted with TAA-Ls or other TAA-targeted therapies, or early relapse may be treated with non-targeted anticancer agents.

Lactate dehydrogenase is associated with germ cell tumors, lymphoma, leukemia, melanoma, and neuroblastoma. RNA encoding Lactate dehydrogenase and other protein markers described herein may be detected using the dd-PCR method of the invention.

Neuron-specific enolase (NSE) is associated with small cell lung cancer and neuroblastoma. RNA encoding NSE and other protein markers described herein may be detected using the dd-PCR method of the invention.

Nuclear matrix protein 22 is associated with bladder cancer. RNA encoding NMP22 and other protein markers described herein may be detected using the dd-PCR method of the invention.

Programmed death ligand 1 (PD-L1) is associated with non-small cell lung cancer. RNA encoding PD-L1 may be detected using the dd-PCR method of the invention.

Prostate-specific antigen (PSA) is associated with prostate cancer. RNA encoding PSA may be detected using the dd-PCR method of the invention.

Thyroglobulin or TG is associated with thyroid cancer. RNA encoding TG may be detected using the dd-PCR method of the invention.

Urokinase plasminogen activator (uPA) and plasminogen activator inhibitor (PAI-1) are associated with breast cancer. RNA encoding uPA may be detected using the dd-PCR method of the invention.

5-Protein signature (OVA1®) markers associated with ovarian cancer. RNA encoding OVA1 protein markers may be detected using the dd-PCR method of the invention.

21-Gene signature (Oncotype DX®) markers associated with breast cancer. RNA encoding these markers may be detected using the dd-PCR method of the invention.

70-Gene signature (Mammaprint®) markers associated with breast cancer. RNA encoding these markers may be detected using the dd-PCR method of the invention.

In addition to the tumor antigen markers described above, other tumor antigen makers including but not limited to: NYO-ESO, HER-2/neu, MAGE-A3, MAGE-A10, CA125, MAGE A4, EPCAM, FOLR1, TPBG, FLT3, MUC 1, ETA, PSA, TERT, MART1, egfr, cd20, gage, and bale may be detected.

Other cellular markers. In addition to tumor or cancer markers, other nucleic acid (i.e., RNA or DNA) markers may be detected and analyzed by the methods disclosed herein including those where dysfunctional proteins are expressed from aberrant mRNA such as Adenosine deaminase 2 (ADA2) deficiency and Farber lipogranulomatosis. Adenosine deaminase 2 (ADA2) deficiency is a disorder characterized by abnormal inflammation of various tissues, particularly the blood vessels (vasculitis). Signs and symptoms can begin anytime from early childhood to adulthood. The severity of the disorder also varies, even among affected individuals in the same family. Farber hpogranulomatosis. Farber lipogranulomatosis is a rare inherited condition involving the breakdown and use of fats in the body (lipid metabolism). In affected individuals, lipids accumulate abnormally in cells and tissues throughout the body, particularly around the joints.

Graft-vs-Host Disease. The method may also be applied in graft-vs-host-disease (“GVHD”) where biomarkers such as ST2 or osteopontin may be simultaneously measured or in autoimmune diseases where markers of T cell proliferation such as TCR beta or T cell regulation such as FOXP3 to simultaneously measure regulatory vs effector function sites. ST2 is a member of the interleukin (IL)-1 receptor family and specifically binds to IL-33. There are 2 functional ST2 isoforms that have opposite roles in innate and adoptive immunity; a transmembrane ST2 forms the complex with IL-33 and induces type 2 immune response and tissue repair. In contrast, soluble ST2 appears to work as a decoy receptor and negatively regulates IL-33 function. Vander Lugt et al, using a proteomic approach, identified high ST2 levels as the biomarker most significantly associated with treatment-refractory GVHD. This single biomarker strongly predicted for TRM when measured either at the onset of GVHD or on day 14 after allogeneic stem cell transplantation (allo-SCT) using either peripheral blood or bone marrow grafts from related or unrelated donors. Osteopontin (OPN), also known as bone sialoprotein I (BSP-1 or BNSP), early T-lymphocyte activation (ETA-1), secreted phosphoprotein 1 (SPP1), 2ar and Rickettsia resistance (Ric), is a protein that in humans is encoded by the SPP1 gene (secreted phosphoprotein 1). OPN has been shown to OPN help initiate and sustain CD8⁺ T cell-mediated GVHD and thus is a potential target in GVHD prevention. FOXP3 (forkhead box P3), also known as scurfin, is a protein involved in immune system responses. A member of the FOX protein family, FOXP3 appears to function as a master regulator of the regulatory pathway in the development and function of regulatory T cells. The methods according to the invention may be used to assess and quantify markers of cancer as well as non-cancer diseases, disorders or conditions, such as those described above, and prognose or guide subsequent treatment.

PCR and dd-PCR The polymerase chain reaction method is used to quantify nucleic acids by amplifying a nucleic acid molecule with the enzyme DNA polymerase. Conventional PCR is based on the theory that amplification is exponential. Therefore, nucleic acids may be quantified by comparing the number of amplification cycles and amount of PCR end-product to those of a reference sample. However, many factors complicate this calculation, creating uncertainties and inaccuracies. These factors include the following: initial amplification cycles may not be exponential; PCR amplification eventually plateaus after an uncertain number of cycles; and low initial concentrations of target nucleic acid molecules may not amplify to detectable levels. However, the most significant limitation of PCR is that PCR amplification efficiency in a sample of interest may be different from that of reference samples. Since PCR is an exponential process, only twofold differences in amplification can be observed, greatly impacting the validity and precision of the results.

dd-PCR improves upon the conventional PCR practices by dividing up the reaction into multiple, smaller reactions. A sample is partitioned so that individual nucleic acid molecules within the sample are localized and concentrated within many separate regions. Micro well plates, capillaries, oil emulsion, and arrays of miniaturized chambers with nucleic acid binding surfaces can be used to partition the samples. A PCR solution is made similarly to a TaqMan assay, which consists of template DNA (or RNA), fluorescence-quencher probes, primers, and a PCR master mix, which contains DNA polymerase, dNTPs, MgCl₂, and reaction buffers at optimal concentrations. The PCR solution is divided into smaller reactions and are then made to run PCR individually. After multiple PCR amplification cycles, the samples are checked for fluorescence with a binary readout of “0” or “1”. The fraction of fluorescing droplets is recorded. The partitioning of the sample allows one to estimate the number of different molecules by assuming that the molecule population follows the Poisson distribution, thus accounting for the possibility of multiple target molecules inhabiting a single droplet. Using Poisson's law of small numbers, the distribution of target molecule within the sample can be accurately approximated allowing for a quantification of the target strand in the PCR product; see Hindson, et al., Anal Chem. 2011 Nov. 15; 83(22): 8604-8610; Vossen, et al., Methods Mol. Biol. 2017; 1492:167-177, or Taylor, et al., Scientific Reports, vol. 7, Article number: 2409(2017); each of which are incorporated by reference.

Probe/Primer. Length of a PCR primer is long enough to cover adequate specificity and short enough for the primer to easily bind to a polynucleotide template, for example, 15, 16, 17, 18, 19, 20, 21, 22, 23, 24, 25, 26, 27, 28, 29 or 30 nucleotides in length. In some embodiments a primer will have a melting temperature of 50, 51, 52, 53, 54, 55, 56, 57, 58, 59 or 60° C. In some embodiments a primer will have a GC content (%) of 30, 40, 50, 60, 70 or 80%. Preferably the Tₘ of two primers used for PCR will not differ by more than 1, 1.5 or 2° C. Preferably primers are designed to not have sequences that form primer dimers or that form hairpin loops.

Primer-BLAST may be used to find primers for amplifying the polynucleotides disclosed herein including those encoding BIRC5 (Survivin), PRAME, and WT1; see hypertext transfer protocol//_www.ncbi.nlm.nih.gov/tools/primer-blast/index.cgi?ORGANISM=9606&INPUT_SEQUENCE=NM_001024210.2&LINK_LOC=nu ccore (last accessed Jun. 25, 2019, incorporated by reference).

Amplicon length is selected based on efficiency of amplification of a target polynucleotide sequence. Usually a short amplicon length is preferred. In some embodiments amplicon length ranges from 50, 60, 70, 80, 90, 100, 110, 120, 130, 140, 150, 160, 170, 180, 190, 200, 210, 220, 230, 240, 250, 260, 270, 280, 290, or 300 nucleotides.

In some embodiments a probe may have a length of 15, 16, 17, 18, 19, 20, 21, 22, 23, 24, 25, 26, 27, 28, 29 or 30 nucleotides and/or have a GC content (%) of 30, 40, 50, 60, 70 or 80%; and/or have a melting temperature of about 5, 6, 7, 8, 9, or 10° C. above than of PCR primers or within a range of 68, 69 or 70° C. Probes and primers having the lengths and other features described above may be designed to one or more portions of a target segment of a TAA polynucleotide or other segments of a TAA polynucleotide suitable for production and identification of an amplicon.

Normalization or relative quantification. The quantity of RNA detected by dd-PCR for a particular tumor associated marker may be normalized to the quantity of RNA detected for a control RNA species, such as ABL1. Relative quantification is based on internal reference genes to determine fold-differences in expression of the target gene. The quantification is expressed as the change in expression levels of mRNA interpreted as complementary DNA (cDNA, generated by reverse transcription of mRNA). Relative quantification is easier to carry out as it does not require a calibration curve as the amount of the studied gene is compared to the amount of a control reference gene. However, in some embodiments, absolute numbers of RNA molecules for different tumor markers may be determined and compared. Other modes of normalization may also be used including those taking into account, adjustments for difference in the type of biological sample used, the kind of probes/primers used to detect nucleic acids encoding TAAs or other assay conditions, or sample collection procedures.

Increase or decrease in expression of a tumor marker RNA may be made relative to a control value for a normal subject, a prior value taken from an undiagnosed subject, or from a subject before or after a cancer therapy. An increase may range from <5, 5, 10, 20, 30, 40, 50, 60, 70, 80, 90, 100% or more relative to a control value and an decrease may range less than 100% of a control value down to 90, 80, 70, 60, 50, 40, 30, 20, 10 or 5% of the control value.

Ratio analysis. The amount or relative expression of RNA of two or more different tumor markers may be compared to produce an expression profile. The expression profile provides information useful to plan cancer treatment (e.g., target a particular cancer marker protein), assess disease progression, determine cancer aggressiveness, and monitor for recurrence. In some embodiments of the invention, targeted therapy may be changed or redirected when a ratio of BLRC5, PRAME, or WT1 increases with respect to a control gene or with respect to another tumor associated antigen. For example, when a normalized ratio of BIRC5/ABL1 increases compared to that of PRAME/ABL1 or WT1/ABL1, then an immunotherapy may be redirected or increased to target BIRC5; when a normalized ratio of PRAME/ABL1 increases compared to that of BIRC5/ABL1 or WT1/ABL1, then an immunotherapy may be redirected or increased to target PRAME: and when a normalized ratio of WTL/ABL1 increases compared to that of BIRC5/ABL1 or PRAME/ABL1, then an immunotherapy may be redirected or increased to target WT1. Similarly, when such ratios fall, then targeted immunotherapy directed to a TAA with a falling ratio may be decreased or terminated.

Ratios relative to other TAAs of a nucleic acid encoding BIRC5, PRAME or WT1 to a nucleic acid encoding another TAA (including BIRC5, PRAME or WT1) may range from 1:1, 1.11, 1.2:1, 1.3:1, 1.4:1, 1.5:1, 1.6:1, 1.7:1, 1.8:1, 1.9:1 2:1, 2.2:1, 2:5:1, 3:1, 3.2:1, 3.5:1, 4:1, 4.2:1, 4.5:1, 5:1, 6:1, 7:1, 8:1, 9:1 or >10:1 as well as all intermediate ratios within this range. When measured at different points in time an elevation of the normalized amount of nucleic acid encoding a particular TAA or a decline in the ratio, will help guide future targeted treatment.

In some embodiments, elevation of a normalized amount of nucleic acid encoding a TAA will indicate or prognose a relapse of a cancer, for example, an increase in the normalized value of BIRC5/ABL RNA in a biological sample when measured at different points in time. In other embodiments, increases in two, three of more of normalized ratios of BIRC5, PRMAE, WT1 or other TAAs will indicate or prognose a more intractable cancer.

Minimal residual disease or MDR may be determined by genetic or phenotypic analysis. Standard detection methods define minimal residual disease as an incidence of less than one cancer, tumor or neoplastic cell in 10,000 normal cells. In some cases, minimal residual disease may exhibit an incidence of less than one cancer cell in 5,000, 10,000, 20,000, 50,000, 100,000, 200,000 cells or any intermediate value within this range.

Remission is defined as the absence of outward signs of cancer or the absence of detectable cancer cells in the body, for example, after a course of therapy. In some cases spontaneous remission may occur. Remission can be characterized, for example, as a lack of detectable abnormal cells in the blood, bone marrow, and/or cerebrospinal fluid using the dd-PCR method of the invention. Remission may also be characterized by reduced expression or lack of expression of tumor marker RNA compared to that in a control subject without cancer or compared to an earlier control value from the same subject.

Subject refers to a human or animal, including all vertebrates, e.g., mammals such as primates (particularly higher primates), sheep, dog, rodents (e.g., mouse or rat), guinea pig, goat, pig, cat, rabbit, and cow, etc. Typically, the subject is a human and has been diagnosed with cancer using a diagnostic method known in the art. For example, a subject may have been diagnosed with cancer using histopathology, imaging tests, and/or blood tests. In some embodiments a subject will not yet have been diagnosed with cancer. A subject may be no more than 0, 1, 2, 3, 4, 5, 6, 7, 8, 9, 10, 11, 12, 13, 14, 15, 16, 17, 18, 19, 20, 21, 25, 30, 35, 40, 45, 50, 55, 60, 65, 70, 75, 80, 85, 90, 95, or 100; or older than 100 years old.

The invention includes, but is not limited to, the following embodiments.

One embodiment of the invention is a method for determining after a prior cancer treatment a risk of relapse in a subject having minimal residual cancer that includes: (a) obtaining a biological sample from a subject, and (b) detecting a level of BIRC5 (Survivin) RNA in the biological sample, and (c) selecting a subject at risk of relapse of minimal residual cancer when BIRC5 RNA is detected; and, optionally, (d) treating the subject, modifying an ongoing treatment of the subject, or terminating treatment of the subject. This method may be performed on a subject who has been previously treated for leukemia, lymphoma, myeloma, or another blood disorder. A biological sample used in this method may be any sample containing RNA encoding a tumor associated antigen such as BIRC5. Some nonlimiting types of biological samples include blood, buffy coat cells, plasma, cell free plasma, or serum. In a preferred embodiment, this method is performed using a digital drop polymerase chain reaction (“ddPCR”) with probes or primers recognizing a target segment of at least one polynucleotide encoding BIRC5, PRAME or WT1. In some alternative embodiments, a level of RNA encoding a non-BIRC5 tumor associated antigen may be determined, such as RNA encoding PRAME or WT1. In other embodiments, a level of BIRC5 encoding RNA as well as one or more levels of RNAs encoding other tumor associated antigens may be determined, for example, by a multiplex dd-PCR procedure.

This method may further include detecting the level of BIRC5 RNA over at least two, three, four or more different points in time and selecting a subject at risk of relapse and/or in need of treatment when BIRC5 RNA levels increase over time. In alternative embodiments levels of RNAs encoding other tumor associated antigens may be determined over two, three, four or more different points in time. An interval between different time points may range from 1, 4, 8, 12, 16, 20, or 24 hours; 1, 2, 3, 4, 5, 6, or 7 days, 1, 2, 3, 4, 5, 6, 7, or 8 weeks, or 1, 2, 3, 4, 5, 6, 7, 8, 9, or 12 months, or 1, 2, 3, 4, 5, 6, 7, 8, 9 or 10 years. An interval of time between different points of detecting RNA encoding tumor associated antigens may be selected by those skilled in the art based on patient status, type of anticancer treatment being administered to a patient, or one the expected duration of therapy.

The method described above may further include detecting over at least two, three, four or more different points in time a level of PRAME RNA and selecting a subject at risk of relapse and in need of treatment when PRAME RNA levels increase over time; and/or detecting over at least two, three, four or more different points in time a level of WT1 RNA and selecting a subject at risk of relapse and in need of treatment when WT1 RNA levels increase over time; or detecting levels of WT1 RNA and PRAME RNA, and selecting a subject at risk of relapse and in need of treatment when one, two or three of BIRC5, WT1, or PRAME RNAs are detected or when levels or RNAs encoding these tumor associated antigens increase, stay constant or decrease. This method may also include detecting a level of at least one of ABL, BCR, GUSB or other control gene RNA, and normalizing the level of BIRC5, PRAME, WT1 RNA levels (or other tumor associated antigen RNA levels) to the level of one or more control genes.

This method may be performed on samples from a subject who was previously treated with surgery, radiation, chemotherapy, cytokine, BCG, monoclonal antibodies, T cells, or other immunotherapy, including antibody- or T cell-based immunotherapies that target one or more tumor associated antigens such as BIRC5, PRAME or WT1. In other embodiments, the method may be performed on a subject

In some embodiments, a subject who was previously treated with a tumor-antigen targeted chemotherapy or tumor antigen targeted immunotherapy, such as with antibodies or T cells that bind to or otherwise recognize tumor associated antigens or cells expressing them. Thus, a subject may have been previously treated with TAA-L to at least one tumor-associated antigen of a leukemia, lymphoma or other blood disorder; or previously treated for leukemia, acute lymphoblastic leukemia (“ALL”), acute myeloid leukemia (“AML”), chronic lymphocytic leukemia (CLL), chronic myelogenous leukemia (CML), hairy cell leukemia, mast cell leukemia, or meningeal leukemia, Hodgkin's lymphoma, anaplastic large-cell lymphoma (“ALCL”), Burkett's lymphoma, splenic marginal zone lymphoma, hepatosplenic T-cell lymphoma, angioimmunoblastic T-cell lymphoma (AILT), or other non-Hodgkin's lymphoma, a myelodysplastic syndrome (“MDS”), or multiple myeloma, Waldenström macroglobulinemia, plasmacytoma or other myeloma.

In other related embodiments, the method may include further treating the subject for minimal residual cancer or early relapsing cancer when BIRC5 is detected, further treating the subject with TAA-L or another chemo- or immunotherapy that targets BIRC5 when BIRC5 is detected, further treating the subject with TAA-L or another chemo- or immunotherapy that targets PRAME when PRAME is detected, further treating the subject with TAA-L or another chemo- or immunotherapy that targets WT1 when WT1 is detected, further treating the subject with TAA-L or another chemo- or immunotherapy that targets BCR-ABL when BCR-ABL is detected, further treating the subject into remission of a leukemia, lymphoma, or other blood disorder, prior to detecting a level of BIRC5.

Treatments include, but are not limited to use of microRNA that regulates expression of BIRC5, PRAME, or WT1 which may be administered to, or induced in, a subject expressing abnormal levels of RNA encoding these antigens, such as abnormally high levels of BIRC5.

Examples of microRNA that target BIRC5 include miR-16, miR-34a, miR-143, miR-150, miR-203, mir-218, miR-320a, miR-494, miR-542-3p and miR-708. Delivery methods for miRNA include both viral-based and non-viral based systems, such as polyethylenimine or lipid-based delivery systems; see Zhang Y, et al., J Control Release. 2013 Dec. 28; 172(3):962-74; and Miso, et al., Mol Ther Nucleic Acids. 2014 Sep. 23; 30:e194 (both incorporated by reference).

Other therapies that target BIRC5 include administration of YM155 (Sepantronium Bromide) which is a small molecule suppressant of the survivin promoter; and administration of LY2181308 which targets BIRC5 mRNA.

In some embodiments, in conjunction with determining a level of BIRC5/Survivin RNA, levels of one or more miRNAs that target BIRC5/Survivin may be determined by methods known in the art, where lower than normal levels of one or more BIRC5 targeting miRNAs in conjunction with a higher level of BIRC5 RNA correlate with a higher risk of relapse. Examples of microRNA that target BIRC5 include miR-16, miR-34a, miR-143, miR-150, miR-203, mir-218, miR-320a, miR-494, miR-542-3p and miR-708.

The method as disclosed herein may be performed on biological samples obtained from subjects of different genetic backgrounds (including major and minor histocompatibility antigens), or ages. Such samples may be obtained from subjects no more than, or at least, 0, 1, 2, 3, 4, 5, 6, 7, 8, 9, 10, 11, 12, 13, 14, 15, 16, 17, 18, 19, 20, 21, 25, 30, 35, 40, 45, 50, 55, 60, 65, 70, 75, 80, 85, 90, 95, 100 or >100 years old (or any intermediate value or subrange). For example, in some embodiments a subject may be no more than five years old, in others more than five years old but no more than 16 years old, and in others at least 16 years old.

Another embodiment of the invention is a kit for determining a risk of relapse in a subject having minimal residual disease of cancer comprising probes and/or primers that amplify BIRC5 (Survivin), PRAME, WT1 and/or BCR-ABL RNA such as Taqman-type probes/primers; and, optionally, RNA purification equipment, reaction containers or substrates where an RNA sample and probes or primers are combined, packaging materials, and/or instructions for use. The kit may also include probes/primers that amplify or detect ABL, BCR, GUSB, or one or more other control genes.

Another embodiment of the invention is a method for monitoring a subject who has undergone cancer treatment including (a) obtaining a biological sample from the subject, and (b) detecting by dd-PCR multiplexing the presence or absence of a level of RNA of least three different antigens associated with said cancer; and (c) selecting a subject whose cancer has progressed, been stable, or regressed based on relative levels of said at least three different antigens detected by multiplexing; and, optionally, (d) treating the subject, modifying an ongoing treatment of the subject, or terminating treatment of the subject. In this method a subject may be a leukemia or lymphoma patient, the biological sample may be blood, buffy coat cells, plasm, cell free plasma, or serum, and detecting may include detecting a level of BIRC5, PRAME and WT1 RNA.

Another embodiment of the invention is a method for treating a subject having cancer including (a) treating a subject for cancer, (b) after said treatment (a), detecting using dd-PCR a level of one or more cancer antigens in a biological sample obtained from the subject, and (c) retreating the subject with a therapy to one or more cancer antigens detected by the dd-PCR; and, optionally, (d) repeating (b) and/or (c). In this method in (a) a subject may be treated into remission of the cancer or into a state of minimal residual disease; the biological sample may be blood, buffy coat cells, plasma, cell free plasma, or serum.

In some embodiments of this method in (a) or (c), the treating or retreating involves infusing or otherwise transferring into the subject TAA-L that recognize at least one cancer associated antigen, such as BIRC5, PRAME or WT1; or in (a) or (c), the treating or retreating involves infusing or otherwise transferring into the subject a drug that selectively targets at least one cancer antigen or a functional activity of at least cancer antigen.

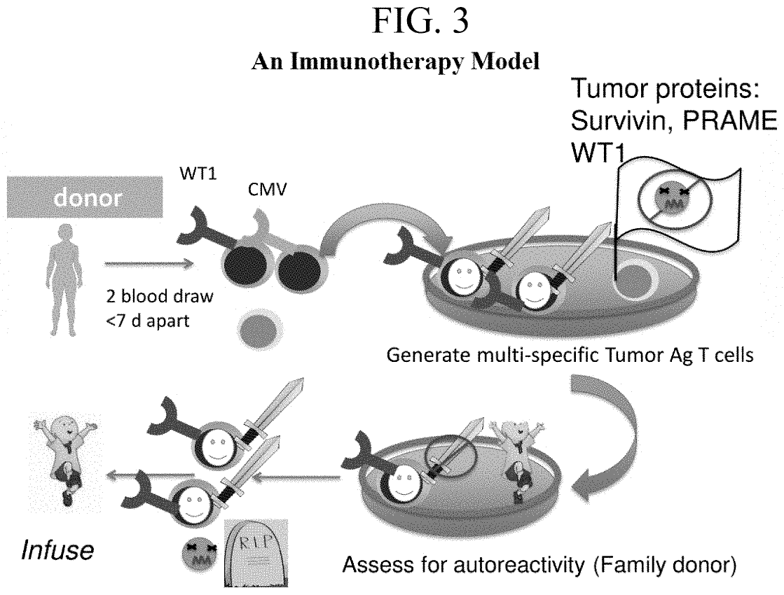

Methods for producing TAA-L are disclosed by and incorporated by reference to U.S. 2018/0072990-A1. TAA-Ls to BIRC5, PRAME, WT1 and other TAAs may be produced using naïve cells, stem cells, or other types of donor cells. This method may use donor cells that are fully histocompatible with or autologous to the patient. A donor may also be partially histocompatible or allogeneic with a patient sharing one, two, three, four, five, six, seven, eight, nine or ten HLA markers, such as HLA-A, HLA-B, HLA-C, HLA-DR, HLA-DQ or HLA-DP types with the patient. A donor may be fully matched, such as an identical twin, or at least half-matched (haploidentical) with a patient, for example, a donor may be a parent or child of a patient. When the donor T cells are not autologous, they can be screened to remove autoreactive T cells. Once produced, TAA-L can be infused into a patient to attack cancer cells expressing the target antigen such as BIRC5, PRAME or WT1. One method for producing TAA-L is described by FIG. 3 where donor T cells are contacted with tumor antigens such as PRAME, BIRC5 (Survivin), and WT1 to generate multispecific population of T cells recognizing these antigens.

In some embodiments of this method, in (b) BIRC5 is detected and (c) comprises treating the subject with an immunotherapy targeted at BIRC5; in (b) PRAME is detected and (c) comprises treating the subject with an immunotherapy targeted at PRAME; in (b) WT1 is detected and (c) comprises treating the subject with an immunotherapy targeted at WT1; in (b) a level of BIRC5, PRAME, or WT1 RNA is absent or is lower than a pretreatment baseline level and therapy targeted at the absent antigen or antigen present at a lower than baseline level is reduced or terminated in (c).

Another embodiment of the invention is directed to method for assessing disease progression in a subject having cancer or other disease, disorder or condition characterized by abnormal proliferation, aberrant RNA or abnormal RNA levels including (a) obtaining a biological sample from the subject, (b) identifying one or more cancer antigen RNAs, aberrant RNA or RNs present at an abnormal level, (c) repeating (a) and (b) at a different time point, (d) comparing a level of said one or more RNAs identified in (b) and (c), and (e) selecting a subject whose disease has progressed based on higher or farther from normal levels of said identified RNAs or selecting a subject whose disease has regressed based on lower or closer to normal levels of said identified RNAs; and (f) and optionally, treating said subject whose disease has progressed, modifying an ongoing treatment, or reducing or terminating treatment for a subject whose disease has regressed. In this method the biological sample may be blood, buffy coat cells, plasma, cell free plasma, or serum. In this method one or more RNAs may be BIRC5, PRAME and/or WT1 RNA.

Another embodiment of the invention involves a method for monitoring for recurrence or a cancer including (a) obtaining a biological sample from a subject, (b) identifying one or more cancer antigen RNAs in the RNA from the biological sample, or identifying the absence of said RNA in the biological sample, (c) selecting a subject whose disease has recurred when said one or more RNAs are detected in the biological sample, or selecting a subject whose disease has not recurred when said one or more RNAs is not detected in the biological sample; and, optionally, (d) treating said subject whose disease has recurred, modifying treatment, or reducing or terminating treatment for a subject whose disease has not recurred. In this method the biological sample may be blood, buffy coat cells, plasma, cell free plasma, or serum. In this method a subject may have a disease or have been previously treated for a disease that is leukemia. In some embodiments, the one or more RNAs is BIRC5, PRAME and/or WT1 RNA. For example, the disease may be leukemia and the one or more RNAs include BIRC5 RNA, an increase of BIRC5 RNA in the biological sample may be measured at two or more points in time, and a subsequent treatment targeting BIRC5 may be increased or initiated. In another example of this method the disease is leukemia and the one or more RNAs includes PRAME RNA, an increase of PRAME RNA in the biological sample as measured at two or more points in time is detected, and a subsequent treatment targeting PRAME is increased or initiated. In other example, the disease is leukemia and the one or more RNAs comprises WT1 RNA, an increase of WT1 RNA in the biological sample as measured at two or more points in time is detected, and a subsequent treatment targeting WT1 is increased or initiated.

Another embodiment of the invention is directed to method for monitoring for progression of a cancer that includes (a) obtaining a biological sample from a subject, (b) detecting a level of BIRC5, PRAME, and WT1 RNA in said sample at a base line time point, (c) detecting a level of BIRC5, PRAME, and WT1 RNA in said sample at one or more points in time after the base line time point, (d) selecting a subject whose disease has progressed when said one or more of BIRC5, PRAME, or WT1 RNA levels has increased or selecting a subject whose disease has not progressed when said one or more levels have not increased, (d) treating the subject whose disease has progressed, modifying the treatment, or maintaining, reducing or terminating treatment for a subject whose disease has not progressed. In this method the biological sample is blood, buffy coat cells, plasma, cell free plasma, or serum. In some embodiments of this method the cancer is leukemia or lymphoma, the subject is in clinical remission or in a state of minimal residual disease, and a subject with disease progression is selected when an increased amount of BIRC5 compared to a baseline value is detected; or wherein a baseline level of BIRC5 RNA is lower than a prior baseline and a WT1 RNA and/or PRAME RNA level is higher. For example, the cancer may be leukemia or lymphoma, the subject is not in clinical remission, or in a state of minimal residual disease, and a subject with disease progression is selected wherein a greater number of tumor associated antigens are detected (c) than in (b). In another example, the cancer is leukemia or lymphoma, the subject is not in clinical remission, or not in a state of minimal residual disease, and a subject with no or decreased disease progression is selected wherein a greater number of tumor associated antigens are detected (b) than in (c).

This method may further include treating the subject with at least one therapy that targets BIRC5, PRAME or WT1 or that reduces the level of, or the number of, tumor associated antigens detected.

Another embodiment of the invention is a method for determining a risk of relapse in a subject having minimal residual cancer including (a) obtaining a biological sample comprising living cells from a subject, (b) culturing the sample ex vivo or in vitro and exposing the cultured sample to at least one anticancer treatment, (c) detecting a level of BIRC5 (Survivin), WT1 and/or PRAME RNA in the cultured biological sample after said treatment, and (d) selecting a subject at risk of relapse of minimal residual cancer when BIRC5, WT1 or PRAME RNA is detected in the cultured sample. In some embodiments of this method a sample is obtained from a subject previously treated for cancer; or a sample is obtained from a subject who initially (e.g., before a first cancer treatment, after a first cancer treatment, or during a period of remission) did not express at least one of BIRC5, WT1 or PRAME or initially did not express BIRC5, WT1 and PRAME. In other embodiments of this method the culturing ex vivo or in vitro includes contacting the sample with at least one agent that accelerates cell division or growth, such as a medium enriched for factors preferentially promoting growth of cancer cells, one or more cytokines, one or more growth factors, or one or more mitogens such as PHA, conconavalin A, LPS or pokeweed mitogen. In other embodiments, the cells are cultured in the presence of an anticancer drug or agent, antibodies to tumor associated antigens, such as antibodies that bind to BIRC5, PRAME or WT1 or T cells that recognized tumor associated antigens such as T cells recognizing BIRC5, PRAME or WT1. The exposure of the cultured cells grown ex vivo or in vitro may be lower, the same as, or higher that doses of anticancer drugs, biologics or other agents occurring in vivo during cancer treatment of a patient, for example, the concentration may range from 0.1, 0.25, 0.5, 1, 1.5, 2, 3, 4 or 5 times that occurring in vivo in the blood or in or around cancer cells during cancer treatment. In other embodiments of this method the selecting a subject at risk of relapse involves detecting BIRC5 RNA or a higher than initial level of BIRC5 RNA in the sample cultured ex vivo or in vitro.

### Example

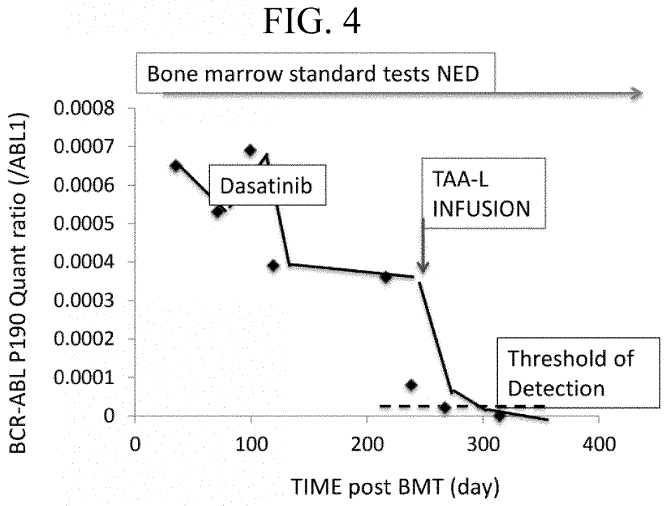

The quantity of a TAA marker was measured before and after an anti-cancer drug treatment. As shown by FIG. 4, the quantity of BCR-ABL marker declined after Dasatinib treatment below a threshold of detection. However, this test would fail to detect residual disease or relapse of a tumor that no longer expresses detectable BCR-ABL and fail to detect the relative levels of other TAAs that could serve as future targets for immunotherapy.

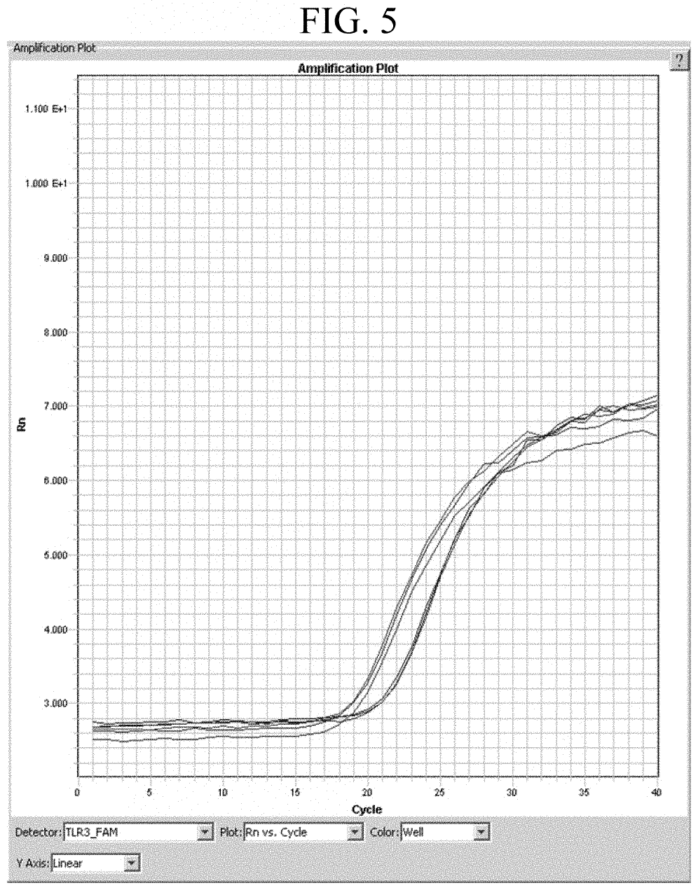

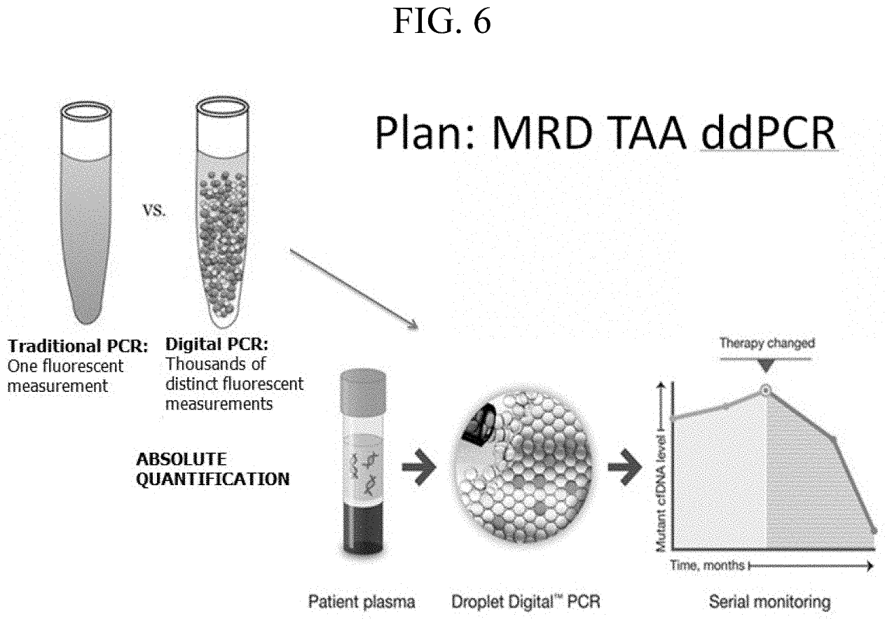

FIG. 5 shows that a reverse-transcriptase procedure can detect individual TAAs and FIG. 6 shows that dd-PCR can be used to detect tumor antigen RNA in plasma and direct further immunological treatment.

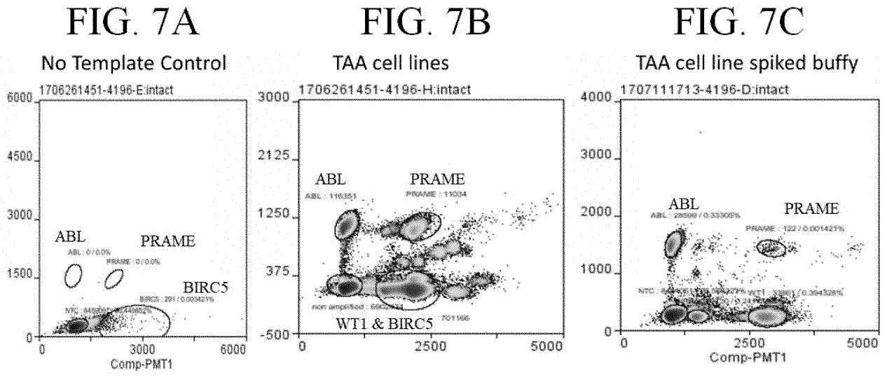

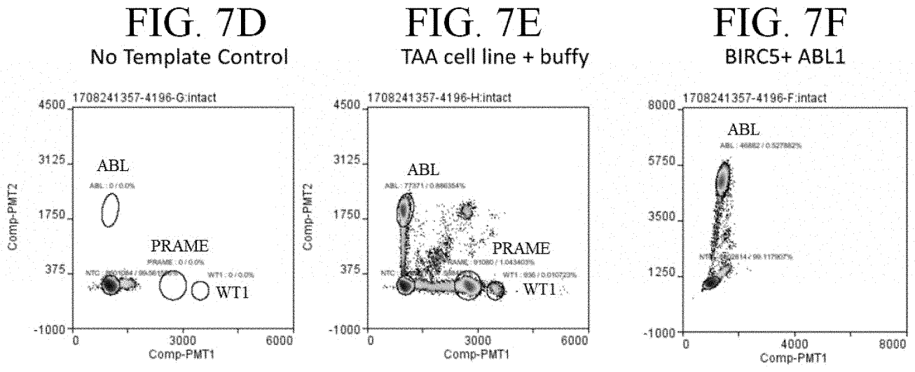

To help lay a foundation for a successful design of a multiplexed dd-PCR that could simultaneously detect and quantify BIRC5, PRAME, WT1 and other TAAs, a multiplexed Taqman assay was evaluated. A control gene, ABL, was assigned its own color. Results showed that cell lines expressing PRAME, WT1 and BIRC5 (Survivin) tumor associated antigens as well as normal buffy coat cells spiked with these cancer cells could be simultaneously detected and that lower input doses yielded better results (FIG. 7B). However, detection of PRAME was lost in the sample of spiked buffy coat cells (FIG. 7C). To improve sensitivity and specificity it was decided to build specific probe/primer combinations to detect each TAA.

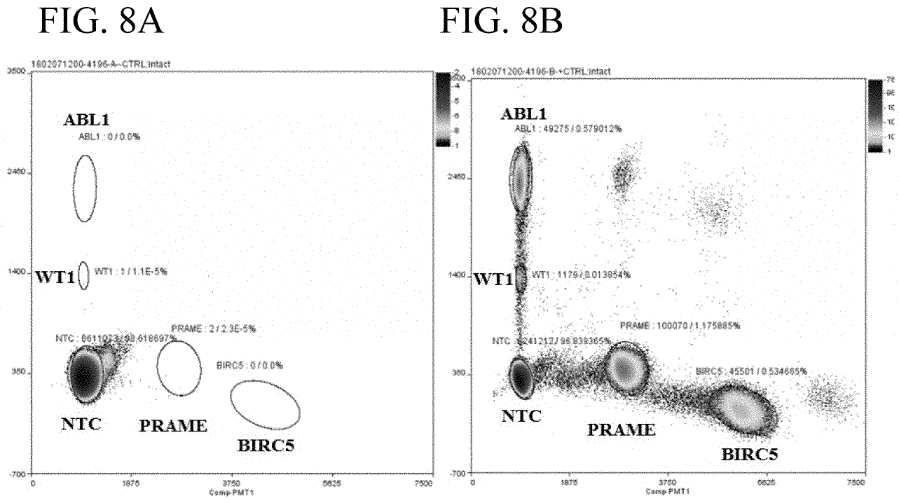

As shown by FIGS. 8A, 8B and 8C, when specific probe/primer combinations were used for dd-PCR detection of BIRC5, PRAME, and WT1, probe specificity, cluster separation, and amplification efficiency were improved and highly conserved regions were detected. However, BIRC5 was not detected (FIG. 8C). While not being bound to any particular theory or explanation, the inventors believe that this may have been due to hairpin binding with another probe/primer even though BIRC5 was the only TAA not detected because BIRC5 probes/primers worked using conventional PCR but did not detect BIRC5 using qPCR. Consequently, new probes/primers were designed.

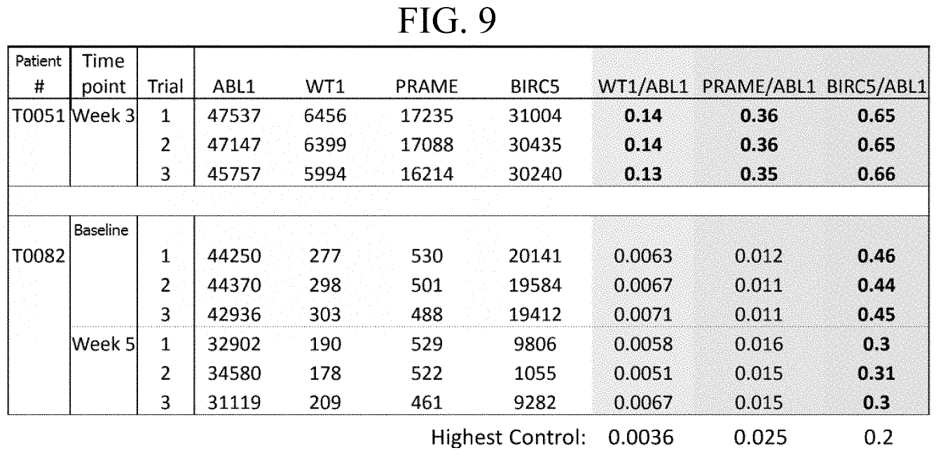

As shown by FIG. 9A (no template, control) and FIG. 9B (positive control), new probes/primers detected BIRC5 (Survivin), PRAME, WT1, and ABL1. As shown by FIG. 9 these probes/primers yielded reproducible results and background readings from eight healthy controls were low within the ranges 0.01 to 0.2 for BIRC5 (Survivin), 0.0005-0.025 for PRAME and 0.0019-0.0036 for WT1.

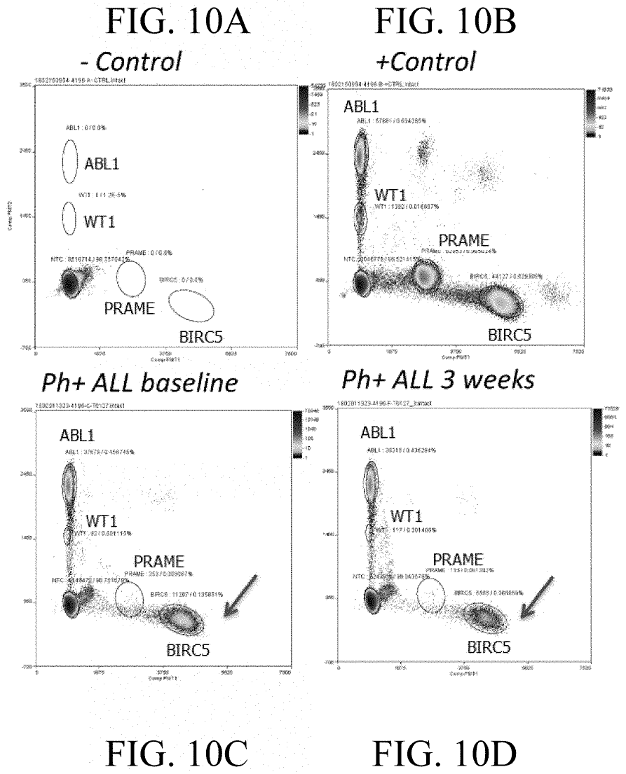

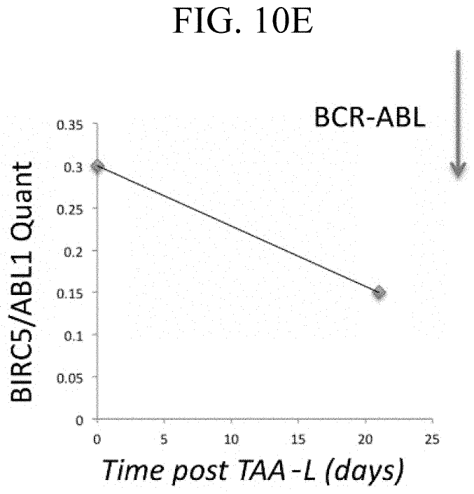

FIGS. 10A and 10B show control values; as shown by FIGS. 10C and 10D show that the primers/probes could detect a decrease in level of RNA encoding BIRC5.

To determine whether results would correlate with results obtained from clinical tests, dd-PCR results (FIGS. 10A-10D) were compared to CLIA certified BCR-ABL results (FIG. 10E). A decrease in BIRC5 was observed over a period of three weeks which mirrored decrease in BCR-ABL.

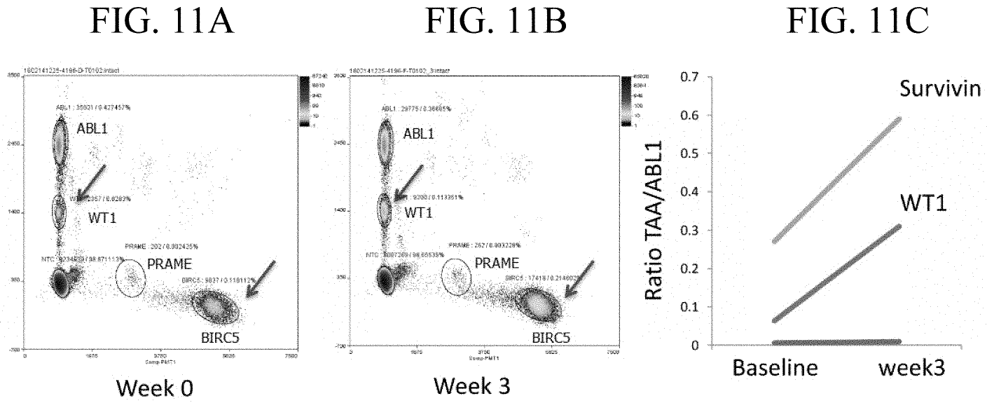

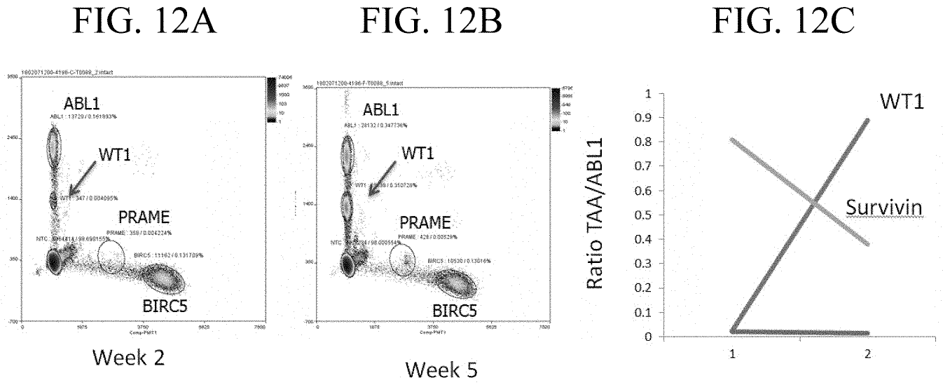

The dd-PCR method also tracked clinical progressive disease as shown by FIGS. 11A-11C. Tested patient was in clinical remission at baseline but had falling blood cell counts (a sign of AML) and no test (bone marrow). These data show an increase in WT1 and BIRC5 (Survivin) associated with relapse or progressive disease. After immunotherapy, the tumor acquired WT1 and downregulated BIRC5 (Survivin). Such data characterized whether a subject's remission was stable or is starting to relapse. As shown by these data, loss of expression of BIRC5 and increased expression of WT1 correlated with relapse. These data also guide treatment of a patient beginning to relapse by revealing TAA targets on relapsing cancer cells.

These data as well as others showed that all patients who had clinical progression exhibited progression as determined by ddPCR and that all patients who had clinical responses to treatment exhibited responses as determined by ddPCR. These results show the excellent positive predictive value and sensitivity of the ddPCR based method of the invention for assessing clinical therapy and/or characterizing disease progression. For example, based on these data a patient may be treated for relapse prior to developing the usual symptoms of a relapsing cancer, and/or a new or retargeted immunotherapy directed at WT1 may be initiated.

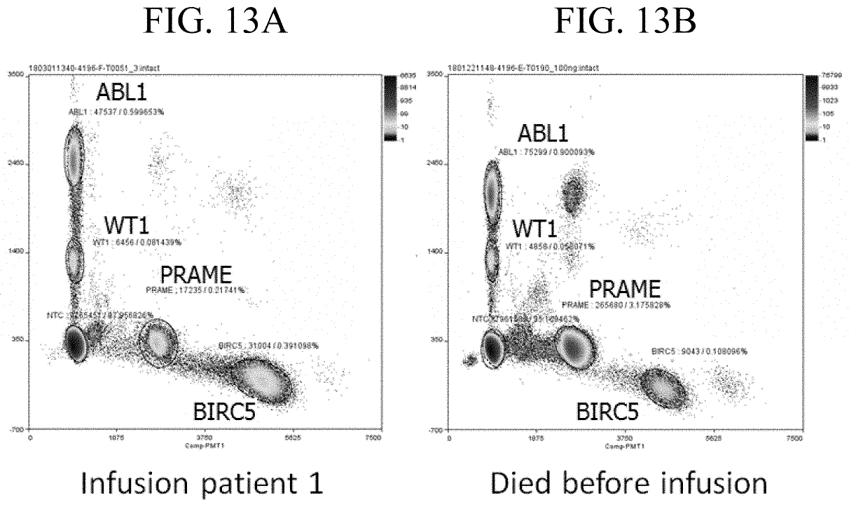

Further work showed that increases of two or more of the BIRC5, PRAME, and WT1 markers correlated with advancing disease in a patient having advanced disease. As shown by comparison of FIGS. 13A and 13B prior to death this patient showed higher levels of PRAME. This and other work shows that advanced disease is characterized by high expression of all the TAAs, that cancers expressing two TAAs are more advanced than those expressing one TAA and that as patients advance they tend to acquire higher levels of WT1. These results show the excellent ability of the dd-PCR methods using BIRC5, PRAME, and WT1 to follow disease progression even in late stage disease.

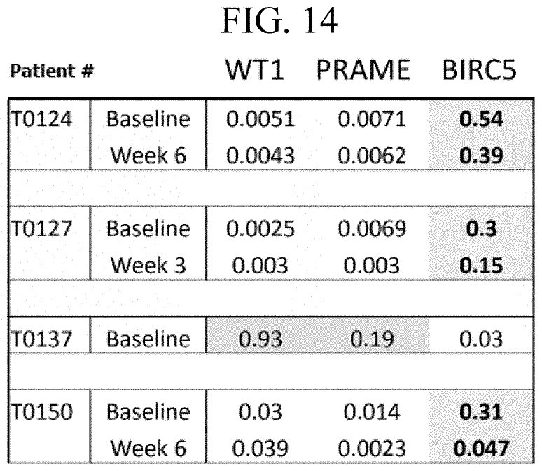

Results from a dd-PCR method according to the invention found that BIRC5 (also known as Survivin) can serve as a marker of minimal residual disease in leukemia. BIRC5 has not previously been evaluated as a biomarker for leukemia. Most of cancer patients evaluated by the inventors were positive for BIRC5. As shown by the data in FIG. 14 all four patients expressed BIRC5 at baseline within the range 0.03 to 0.54. Three to six weeks after baseline, BIRC5 levels declined compared to baseline. As disclosed herein, BIRC5 expression has been found to follow responses to disease therapy.

Terminology. Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

Links are disabled by deletion of http: or by insertion of a space or underlined space before www. In some instances, the text available via the link on the “last accessed” date may be incorporated by reference.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1-66. (canceled)

67. A method for monitoring for progression, relapse, or recurrence of a cancer comprising:
(a) obtaining a biological sample from a subject,
(b) detecting a level of BIRC5, PRAME, and WT1 RNA in said sample at a base line time point using digital droplet polymerase chain reaction (dd-PCR),
(c) detecting a level of BIRC5, PRAME, and WT1 RNA in said sample at one or more points in time after the base line time point,
(d) selecting a subject whose disease has progressed, relapsed or recurred when said one or more of BIRC5, PRAME, or WT1 RNA levels has increased, or selecting a subject whose disease has not progressed when said one or more levels have not increased,
(e) treating said subject whose disease has progressed, relapsed or recurred; or maintaining, reducing or terminating treatment for a subject whose disease has not progressed or recurred.

68. The method of claim 67, wherein said biological sample is blood, buffy coat cells, plasma or serum.

69. The method of claim 67, wherein in (b) the detecting is performed with digital drop polymerase chain reaction (“ddPCR”) with probes or primers recognizing a target segment of at least one polynucleotide encoding BIRC5 (Survivin) and optionally at least one polynucleotide encoding PRAME or WT1.

70. The method of claim 67, wherein the cancer is leukemia or lymphoma, wherein the subject is in clinical remission or in a state of minimal residual disease, and wherein a subject with disease progression is selected when an increased amount of BIRC5 compared to a baseline value is detected; or wherein a baseline level of BIRC5 RNA is lower than a prior baseline and a WT1 RNA and/or PRAME RNA level is higher.

71. The method of claim 67, wherein the cancer is leukemia or lymphoma, wherein the subject is not in clinical remission, or not in a state of minimal residual disease, and wherein a subject with disease progression is selected wherein a greater number of tumor associated antigens are detected (c) than in (b).

72. The method of claim 67, wherein the cancer is leukemia or lymphoma, wherein the subject is not in clinical remission, or not in a state of minimal residual disease, and wherein a subject with no or decreased disease progression is selected wherein a greater number of tumor associated antigens are detected (b) than in (c).

73. The method of claim 67, further comprising treating the subject with at least one therapy that targets BIRC5, PRAME or WT1 or that reduces the level of, or the number of, tumor associated antigens detected.

74. The method of claim 67, wherein said subject has minimal residual disease.

75. A method for treating a subject having cancer comprising:
(a) treating a subject for cancer,
(b) after said treatment (a), detecting using dd-PCR a level of one or more cancer antigens in a biological sample obtained from the subject, and
(c) retreating the subject with a therapy to one or more cancer antigens detected by the dd-PCR; and, optionally,
(d) repeating (b) and/or (c).

76. The method of claim 75, wherein in (a), the subject has been treated into remission of the cancer or into a state of minimal residual disease.

77. The method of claim 75, wherein the biological sample is blood, buffy coat cells, plasma or serum.

78. The method of claim 75, wherein in (b) the detecting is performed with digital drop polymerase chain reaction (“ddPCR”) with probes or primers recognizing a target segment of at least one polynucleotide encoding BIRC5 (Survivin) and optionally at least one polynucleotide encoding PRAME or WT1.

79. The method of claim 75, wherein in (a) or (c), the treating or retreating comprises infusing or otherwise transferring into the subject TAA-L that recognize at least one cancer antigen.

80. The method of claim 75, wherein in (a) or (c), the treating or retreating comprises infusing or otherwise transferring into the subject a drug that selectively targets at least one cancer antigen or a functional activity of at least cancer antigen.

81. The method of claim 75, wherein the cancer is leukemia, lymphoma, myeloma, or another blood disorder.

82. The method of claim 75, wherein the cancer expresses BIRC5, PRAME, and/or WT1 and the therapy in (c) targets BIRC5, PRAME and/or WT1.

83. The method of claim 75, wherein in (b) BIRC5 is detected and (c) comprises treating the subject with an immunotherapy targeted at BIRC5.

84. The method of claim 75, wherein in (b) PRAME is detected and (c) comprises treating the subject with an immunotherapy targeted at PRAME.

85. The method of claim 75, wherein in (b) WT1 is detected and (c) comprises treating the subject with an immunotherapy targeted at WT1.

86. The method of claim 75, wherein in (b) a level of BIRC5, PRAME, or WT1 RNA is absent or is lower than a pretreatment baseline level and therapy targeted at the absent antigen or antigen present at a lower than baseline level is reduced or terminated in (c).


SEQUENCE LISTING (4 sequences)

SEQ ID NO: 1 - DNA, 840 bp, Homo sapiens
  misc_feature (1)..(840): Gene name ABL1.  Transcript Targets NM_005157.5, NM_007313.2
  misc_feature (89)..(667): Probe/primer target segment
ttcatgacctacgggaacctcctggactacctgagggagtgcaaccggcaggaggtgaacgccgtggtgctgctgtacatggccactcagatctcgtcagccatggagtacctggagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgacctgtcccaggtgtatgagctgctagagaaggactaccgcatggagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgagtaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaag

SEQ ID NO: 2 - DNA, 848 bp, Homo sapiens
  misc_feature (1)..(848): Gene name BIRC5.  Transcript Targets NM_001012270.1, NM_001012271.1, NM_001168.2
  misc_feature (92)..(826): Probe/primer target segment.
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggag

SEQ ID NO: 3 - DNA, 920 bp, Homo sapiens
  misc_feature (1)..(920): Gene name WT1.  Transcript Targets NM_000378.4, NM_001198551.1, NM_001198552.1, NM_024424.3, NM_024426.4.
  misc_feature (178)..(822): Probe/primer target segment
ctcactccttcatcaaacaggagccgagctggggcggcgcggagccgcacgaggagcagtcttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggt

SEQ ID NO: 4 - DNA, 840 bp, Homo sapiens
  misc_feature (18)..(742): Probe/primer target segment
gccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatct